(19) United States
(12) Patent Application Publication
(10) Pub. No.: US 2025/0186445 A1
(43) Pub. Date: Jun. 12, 2025

(54) TREATMENT OF CANCER WITH MENIN INHIBITORS AND IMMUNO-ONCOLOGY AGENTS

(71) Applicants: University Health Network, Toronto (CA); Kura Oncology, Inc., San Diego, CA (US)

(72) Inventors: Housheng HE, North York, Ontario (CA); Ming Sound TSAO, Toronto, Ontario (CA); Peiran SU, Toronto, Ontario (CA); Francis BURROWS, Solana Beach, CA (US)

(21) Appl. No.: 18/844,007

(22) PCT Filed: Mar. 7, 2023
(86) PCT No.: PCT/US2023/063879

§ 371 Date: Sep. 4, 2024

Related U.S. Application Data
(60) Provisional application No. 63/322,126, filed on Mar. 21, 2022.
(60) Provisional application No. 63/317,903, filed on Mar. 8, 2022.

(51) Int. Cl. A61K 31/519; A61P 35/00; C07K 16/28

(52) U.S. Cl. CPC A61K 31/519; A61P 35/00; C07K 16/2818; C07K 2317/76

(57) ABSTRACT

The present disclosure relates to methods for treating a tumor or a cancer, optionally a solid tumor, in an individual, the method comprising administering to the individual a menin inhibitor and an immuno-oncology agent.

### CROSS-REFERENCE

This application claims the benefit of U.S. Provisional Application No. 63/322,126 filed Mar. 21, 2022, and U.S. Provisional Application No. 63/317,903 filed Mar. 8, 2022, which are all incorporated herein by reference in their entirety.

### BACKGROUND

Tumorigenesis that drives the development and progression of cancer can be generally hallmarked by aberrant cellular processes that lead to, for example, sustained proliferative signaling, a reduced response to growth suppressors, increased resistance to cell death, replicative immortality, and increased angiogenesis. Certain of these immune-mediated processes may promote tumor invasion and metastasis, while others may inhibit tumor progression.

### SUMMARY

Provided herein are methods and compositions based on various discoveries described herein. In one aspect, it was found that expression of MEN1 is enriched in in vivo solid tumor models but not in in vitro solid tumor cell line models, and that MEN1 depletion plays a role in immune system activation. The in vivo-specific enrichment of immune mediators in A549 patient-derived lung cancer cells suggests MEN1 may function through regulating tumor microenvironment interactions, including activation of immune mediators that impact tumor progression. In another aspect, it was found that MEN1 depletion in immunocompetent animal models reduces tumor volume and increases immune activation, while immune-deficient animal models exhibited increased growth rates. In another aspect, it was found that pharmacological inhibition of the menin-MLL protein complex with a menin inhibitor can produce such effects. In another aspect, antitumor or antiproliferative effects can be achieved when combining a menin inhibitor (e.g., Compound I) and an immuno-oncology agent (e.g., a PD-1/PD-L1 axis inhibitor antibody). In addition, as demonstrated herein, a menin inhibitor can induce immune activation, alone or in combination with an immuno-oncology agent. In some aspects, as described herein, effects of an immuno-oncology agent can be improved when combined with a menin inhibitor such as Compound I (e.g., by enhancing the efficacy or immune activation effect of the immuno-oncology agent, or by generating an immune activation effect).

Provided herein are methods of treating a tumor (e.g., a solid tumor) or a cancer (e.g., a liquid cancer such as a hematological cancer or a cancer comprising a solid tumor) in an individual comprising administering to the individual a menin inhibitor and an immuno-oncology agent. Also provided are uses of a menin inhibitor and an immuno-oncology agent in methods of treating a tumor or a cancer comprising administering to the individual the menin inhibitor and the immuno-oncology agent.

Also provided are methods of enhancing an effect, such as a therapeutic effect (e.g., reduction in tumor size, efficacy, reduction in tumor cell growth or proliferation, etc.), of an immuno-oncology agent in an individual comprising administering to the individual the immuno-oncology agent and a menin inhibitor.

Further provided are methods of activating or enhancing an immune response (e.g., increasing the presence, proliferation, or infiltration of immune cells, such as T cells, CD8+ T cells, CD4+ T cells, CD45+ cells, neutrophils, and/or macrophages, increasing expression and/or transcription of double-stranded RNA (dsRNA), increasing cytokine signaling (e.g., expression of cytokines such as CCL4, CXCL1, CXCL8, CXCL9, CXCL10, CD40, IL1B, or IL-33), etc.) in an individual, cell, or sample (such as a cell or sample from the individual) comprising administering to the individual, or contacting a cell or a sample with, a menin inhibitor, optionally in combination with an immuno-oncology agent, optionally wherein the individual has a tumor and/or a cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show data demonstrating that MEN1 depletion (e.g., knockout) promotes proliferation of tumor-derived cell line cells (e.g., A549) in vivo only. FIG. 1A: Cell count increases in A549 cells over time were comparable in sgMEN1 knockout and control (sgLacZ) in vitro. FIG. 1B and FIG. 1C: MEN1 knockouts (FIG. 1B: sgMEN1; FIG. 1C: sgMEN1-12, sgMEN1-53) increased tumor growth in vivo compared to control.

FIGS. 2A-D show data demonstrating that MEN1 expression is inversely correlated with immune cell infiltration (FIG. 2A: CD8+ T cells; FIG. 2B: neutrophils; FIG. 2C: macrophages; FIG. 2D: dendritic cells).

FIGS. 3A-D show data demonstrating that MLL1 expression is positively correlated with immune cell infiltration (FIG. 3A: CD8+ T cells; FIG. 3B: neutrophils; FIG. 3C: macrophages; FIG. 3D: dendritic cells).

FIGS. 4A-B show data demonstrating that MEN1 depletion mediates mouse colon tumor (CT26) growth depending on immune integrity (e.g., MEN1 depletion increases CT26 tumor growth/volume in immunocompromised mice (FIG. 4A), and MEN1 depletion decreases CT26 tumor growth/volume in immunocompetent mice (FIG. 4B)).

FIGS. 5A-D shows data demonstrating that Compound I treatment and genetic knockout of Men1 induces cytokine gene expression in CT26 (FIG. 5A; FIG. 5C) and MC38 (FIG. 5B; FIG. 5D) cells in 2D culture.

FIGS. 6A-D show data demonstrating that Compound I treatment decreases tumor growth (FIG. 6A) and increases CD8+ T cell infiltration (FIG. 6B; FIG. 6C), and that tumor growth decreases were reversed in the presence of an anti-CD8 antibody (FIG. 6D).

FIG. 7 demonstrates and shows the synergistic effect of combining a menin inhibitor (e.g., Compound I) and an immuno-oncology agent (e.g., a PD-1/PD-L1 axis inhibitor antibody) for methods of treating a tumor or cancer.

FIG. 8 shows a mechanism by which a menin inhibitor, such as Compound I, may activate immune cells and reduce tumor growth.

### DETAILED DESCRIPTION

### Definitions

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As also used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain. The terms “C_{x-y} alkenyl” and “C_{x-y} alkynyl” refer to substituted or unsubstituted straight-chain or branched-chain unsaturated hydrocarbon groups that contain at least one double or triple bond respectively. Unless stated otherwise specifically in the specification, a C_{x-y} alkyl, C_{x-y} alkenyl, or C_{x-y} alkynyl is optionally substituted by one or more substituents such as those substituents described herein.

“Carbocycle” refers to a saturated, unsaturated or aromatic ring in which each atom of the ring is a carbon atom. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

“Heterocycle” refers to a saturated, unsaturated, or aromatic ring comprising one or more heteroatoms. Exemplary heteroatoms include N, O, Si, P, B, and S atoms, and preferably N, O, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) p-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryls as defined above which are optionally substituted by one or more substituents such as those substituents described herein.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, and aromatic and non-aromatic substituents of organic compounds. The permissible substituents can be one or more and the same or different for appropriate organic compounds. For purposes of this disclosure, heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic, and a heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Where substituent groups are specified by their conventional chemical formulae, written from left to right, they equally encompass the chemically identical substituents that would result from writing the structure from right to left, e.g., —CH₂O— is equivalent to —OCH₂—.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Enriching for deuterium may afford certain therapeutic advantages, such as increased in vivo half-life and/or exposure, or may provide a compound useful for investigating in vivo routes of drug elimination and metabolism. Isotopically enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E-, and tautomeric forms as well.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Prodrug” is meant to indicate a compound that may be converted under physiological conditions or by solvolysis to a biologically active compound described herein (e.g., compound of Formula (I-A), Formula (I-B), Formula (II-A), Formula (III-A), Formula (IV-A), or Formula (IV-B), or Compound I). Thus, the term “prodrug” refers to a precursor of a biologically active compound that is pharmaceutically acceptable. In some aspects, a prodrug is inactive when administered to an individual but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press) each of which is incorporated in full by reference herein. The term “prodrug” is also meant to include any covalently bonded carriers, which release the active compound in vivo when such prodrug is administered to a mammalian individual. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian individual, cleaves to form a free hydroxy, free amino, or free mercapto group, respectively. Examples of prodrugs include, but are not limited to, acetate, formate, and benzoate derivatives of a hydroxy functional group, or acetamide, formamide, and benzamide derivatives of an amine functional group in the active compound and the like.

As used herein, a “sample” includes and/or refers to any fluid or liquid sample which is being analyzed in order to detect and/or quantify an analyte. In some embodiments, a sample is a biological sample. Examples of samples include, without limitation, a biopsy sample, a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the individual and disease condition being treated, e.g., the weight and age of the individual, the severity of the disease condition, the manner of administration and the like, which can readily be determined by one of ordinary skill in the art. The term also applies to a dose that will induce a particular response in target cells, e.g., reduction in cellular proliferation or an increase in immune cell infiltration or cytokine expression. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual may still be afflicted with the underlying disorder. In certain embodiments, for prophylactic benefit, the compositions are administered to an individual at risk of developing a particular disease, or to an individual reporting one or more of the physiological symptoms of a disease, even though a diagnosis of this disease may not have been made.

As used herein, “efficacy” or “effective” refers to cellular efficacy, in vivo efficacy, therapeutic efficacy (such as anticancer efficacy or antileukemic efficacy), or prophylactic efficacy, as appropriate for the context. In the cellular context, efficacy can be measured by rate of apoptosis, extent of cellular proliferation, gene expression, protein levels, IC₅₀ values, or enzymatic percent inhibition. Increased efficacy may include one or more effects selected from increasing apoptosis, decreasing cellular proliferation, decreasing IC₅₀, and increasing enzymatic percent inhibition. In an in vivo context, such as in animal models, efficacy can be measured by duration of survival or tumor volume. Increased efficacy may therefore include increased duration of or % subject survival, a decrease in the rate of tumor volume increase, a decrease in tumor volume, or a reduction in rate or extent of metastasis. In the clinical or antileukemic context, efficacy can be measured by rates of complete response (CR; can be with or without measurable residual disease, MRD+/−), complete response with partial hematologic recovery (CRh), CR/CRh, complete response with incomplete hematologic recovery (CRi), complete remission with incomplete platelet recovery (CRp), composite complete remission (CRc=CR+CRi (incl. CRp)+CRh), overall response rate (ORR; ORR=CR (MRD+ or MRD−)+CRh+morphologic leukemic-free state (MLFS)), partial response (PR), stable disease without progression (SD), overall survival (OS), blast count levels, duration of remission (DOR), time to relapse, event-free survival, progression-free survival, and other measures. Increase efficacy means that one or more such measures is improved (e.g., increased CR/CRh rate, increased OS, reduced blast count levels, increased ANC).

A “therapeutic effect,” as that term is used herein, encompasses a therapeutic benefit and/or a prophylactic benefit as described above. A prophylactic effect includes delaying or eliminating the appearance or re-appearance of a disease or condition, delaying or eliminating the onset of symptoms of a disease or condition, or slowing, halting, or reversing the progression of a disease or condition, or any combination thereof.

The term “administered with” or “administered in combination with,” and their grammatical equivalents, as used herein, encompass administration of two or more agents to an animal, such as a human, so that both agents and/or their metabolites are present in the individual at the same time. Such administration includes simultaneous administration in separate compositions, administration at different times in separate compositions, or administration in a composition in which both agents are present.

In some embodiments, the menin inhibitor and the immuno-oncology agent may be administered “concurrently,” meaning with overlapping dosing schedules, e.g., at the same time or approximately the same time, or on the same day, or “sequentially,” meaning one after the other in non-overlapping dosing schedules. In some embodiments, administration of the agents may be initiated at different times and then may continue to be administered concurrently (e.g., administration of the menin inhibitor followed by administration of the menin inhibitor and the immuno-oncology agent, or the reverse). In some embodiments, the menin inhibitor continues to be administrated while the immuno-oncology agent administration is stopped or is paused for a period of time.

An “anti-cancer agent,” “anti-tumor agent,” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to an individual by various methods, including intravenous, oral, intramuscular, intraperitoneal, intravesical, subcutaneous, transdermal, buccal, or inhalation or in the form of a suppository.

“Individual” refers to an animal, such as a mammal, for example a human. The methods described herein can be useful in both human therapeutics and veterinary applications. In some embodiments, the individual is a mammal, and in some embodiments, the individual is human. “Mammal” includes humans and both domestic animals such as laboratory animals (e.g., rats, mice) and household pets (e.g., cats, dogs, swine, cattle, sheep, goats, horses, rabbits), and non-domestic animals such as wildlife and the like.

The term “in vivo” refers to an event that takes place in an individual's body.

The term “in vitro” refers to an event that takes places outside of an individual's body. For example, an in vitro assay encompasses any assay run outside of an individual. In vitro assays encompass cell-based assays in which cells alive or dead are employed. In vitro assays also encompass a cell-free assay in which no intact cells are employed.

“Pharmaceutically acceptable carrier or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The terms “antagonist” and “inhibitor” are used interchangeably, and they refer to a compound having the ability to inhibit a biological function (e.g., activity, expression, binding, protein-protein interaction) of a target protein (e.g., menin, MLL1, MLL2, and/or an MLL fusion protein). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

“Signal transduction” or “signaling” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “expression” refers to the process by which a polynucleotide is transcribed into mRNA and/or the process by which the transcribed mRNA (also referred to as a “transcript”) is subsequently translated into peptides, polypeptides, or proteins. If the polynucleotide is derived from genomic DNA, expression may include splicing of the mRNA in a eukaryotic cell. The level of expression (or alternatively, the “expression level”) of a HOXA9 gene can be determined, for example, by determining the level of HOXA9 polynucleotides, polypeptides, and/or gene products (transcripts or encoded polypeptides). A sequence may be overexpressed or underexpressed as compared to the expression level of a reference sample (i.e., a reference level). As used herein, elevated expression levels or overexpression refer to an increase in expression, generally at least 1.25-fold, or alternatively, at least 1.5-fold, or alternatively, at least 2-fold, or alternatively, at least 3-fold, or alternatively, at least 4-fold, or alternatively, at least 10-fold expression over that detected in a reference sample. As used herein, underexpression is a reduction in expression and generally is at least 1.25-fold, or alternatively, at least 1.5-fold, or alternatively, at least 2-fold, or alternatively, at least 3-fold, or alternatively, at least 4-fold, or alternatively, at least 10-fold expression under that detected in a reference sample. Underexpression also encompasses absence of expression of a particular sequence as evidenced by the absence of detectable expression in a test individual when compared to a reference sample.

The term “menin inhibitor” refers to a compound that binds to the menin protein and/or inhibits the protein-protein interaction of menin with a KMT2A(MLL) protein (e.g., MLL1, MLL2, or KMT2A(MLL) fusion protein). In some embodiments, such binding or inhibition is selective. In certain embodiments, the menin inhibitor modulates the menin protein by binding to or interacting with one or more amino acids and/or one or more metal ions. Certain menin inhibitors may occupy the F9 and/or P13 pocket of menin. The binding of a menin inhibitor may disrupt menin or KMT2A(MLL) (e.g., MLL1, MLL2, or a KMT2A(MLL) fusion protein) downstream signaling. In certain embodiments, a menin inhibitor covalently binds menin and inhibits the interaction of menin with MLL. In certain embodiments, a menin inhibitor interacts non-covalently with menin and inhibits the interaction of menin with MLL. In some embodiments, a menin inhibitor has an IC₅₀ of less than 1 μM, or less than 500 nM, or less than 250 nM, or less than 100 nM, in a cellular assay in a cell line with MLL(KMT2A)fusion, such as MV4;11, or in a biochemical assay for MLL(4-43)/menin binding.

The present disclosure provides compounds for modulating the interaction of menin with proteins such as MLL1, MLL2, and KMT2A(MLL) fusion oncoproteins. In certain embodiments, the disclosure provides compounds and methods for inhibiting the interaction of menin with its upstream or downstream signaling molecules including, but not limited to, MLL1, MLL2, and KMT2A(MLL) fusion oncoproteins. Compounds of the disclosure may be used in methods for the treatment of a variety of cancers and other diseases associated with one or more of MLL1, MLL2, KMT2A(MLL) fusion proteins, and menin, such as hematological maligancies.

As used herein, an “immuno-oncology agent” generally refers to and includes an agent that enhances, stimulates, or upregulates an immune response against a cancer in an individual (e.g., in stimulating an immune response for inhibiting tumor growth). In some embodiments, an immuno-oncology agent is a small molecule, antibody, peptide, protein, circular peptide, peptidomimetic, polynucleotide, inhibitory RNA, aptamer, drug compound, or other compound.

### Methods

Provided herein are methods of treating a tumor (e.g., a solid tumor) or a cancer (e.g., a hematological cancer or a cancer comprising a solid tumor) in an individual comprising administering to the individual a menin inhibitor and an immuno-oncology agent. Also provided are uses of a menin inhibitor and an immuno-oncology agent in methods of treating a tumor or a cancer comprising administering to the individual the menin inhibitor and the immuno-oncology agent.

Also provided are methods of enhancing an effect, such as a therapeutic effect (e.g., reduction in tumor size or reduction in growth in tumor size, efficacy, etc.), of an immuno-oncology agent in an individual comprising administering to the individual the immuno-oncology agent and a menin inhibitor.

Further provided are methods of activating or enhancing an immune response (e.g., increasing the presence, proliferation, or infiltration of immune cells, such as T cells, CD8+ T cells, CD4+ T cells, CD45+ cells, neutrophils, and/or macrophages, increasing expression and/or transcription of dsDNA, increasing cytokine signaling (e.g., expression of cytokines such as CCL4, CXCL1, CXCL8, CXCL9, CXCL10, CD40, IL1B, or IL-33, etc.) in an individual, cell, or sample (such as a cell or sample from the individual, such as a biopsy sample or blood or blood component sample) comprising administering to the individual, or contacting a cell or a sample, with a menin inhibitor, optionally in combination with an immuno-oncology agent. In some embodiments, the menin inhibitor increases the presence of immune cells in a tumor sample (e.g., the number of immune cells in a sample, compared to a sample from an individual that did not receive the menin inhibitor or prior to treatment). In some aspects, for the menin inhibitor, the activating or enhancing is measured relative to an individual, cell, or sample prior to menin inhibitor administration or to an individual, cell, or sample that did not receive the menin inhibitor, or to another suitable control, and for a combination of a menin inhibitor and an immuno-oncology agent, the activating or enhancing is relative to the effect of the menin inhibitor alone or relative to the effect of the immuno-oncology agent alone, or to another suitable control. In some embodiments, the sample is a biopsy sample from an individual with a tumor and/or a cancer.

In some embodiments, the menin inhibitor, alone or in combination with an immuno-oncology agent: increases T cell proliferation (e.g., CD8+ T cells) as measured in a Mixed Lymphocyte Reaction (MLR) assay; increases macrophage proliferation; increases CD45+ cell proliferation; increases T cell infiltration (e.g., CD8+ T cells); increases macrophage infiltration; increases neutrophil infiltration; increases expression and/or transcription of cytokine genes (such as CCL4, CXCL1, CXCL8, CXCL9, CXCL10, CD40, IL1B, or IL-33); increases interferon-γ production as measured in an MLR assay; increases cytokine (e.g., IL-2) secretion as measured in an MLR assay; increases immune signaling (e.g., as analyzed in a single-cell rNA-seq or CyTOF assay from a tumor biopsy); increases expression and/or transcription of dsRNA; or inhibits tumor growth in vivo. In some embodiments, the menin inhibitor increases cytokine expression by a cell (e.g., A549 cells, CT26 cells, MC38 cells, or cells in a MLR assay). In some embodiments, for a menin inhibitor alone, the increase or inhibition may be relative to the effect observed with no treatment (or prior to treatment) or in a suitable control, or for a combination of a menin inhibitor and an immuno-oncology agent, the increase or inhibition may be relative to the effect observed with the menin inhibitor alone or relative to the effect observed with the immuno-oncology agent alone, or in a suitable control.

In some embodiments, the immuno-oncology agent comprises a PD-1/PD-L1 axis inhibitor. In some embodiments, the immuno-oncology agent comprises a PD-1/PD-L1 axis inhibitor, a CTLA4 inhibitor, a TIGIT inhibitor, a VISTA inhibitor, a LAG-3 (lymphocyte activation gene 3) inhibitor, a CD73 inhibitor, a CD137 (4-1BB) agonist, an OX40 agonist, a CD40 agonist, a CD27 agonist, a TLR7 agonist, an interferon alpha polypeptide, or an IL-2 polypeptide, or any combination thereof.

In some embodiments, the PD-1/PD-L1 axis inhibitor is an antibody. In certain embodiments, the PD-1/PD-L1 axis inhibitor comprises an anti-PD-1 or anti-PD-L1 antibody. In certain embodiments, the anti-PD-1 antibody or anti-PD-L1 antibody is nivolumab, pembrolizumab, cemiplimab, atezolizumab, dostarlimab, durvalumab, or avelumab.

In some embodiments, the immuno-oncology agent is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587.

In some embodiments, the PD-1/PD-L1 axis inhibitor is MEDI0608 (also known as AMP-514), which is a monoclonal antibody. MEDI0608 is described, for example, in U.S. Pat. No. 8,609,089B2.

In some embodiments, the PD-1/PD-L1 axis inhibitor is pidilizumab (CT-011), which is a humanized monoclonal antibody. Pidilizumab is described in U.S. Pat. No. 8,686,119 B2 or WO 2013/014668 A1.

In some embodiments, PD-1/PD-L1 axis inhibitor antibodies useful in the methods described herein also include isolated antibodies that bind specifically to human PD-1 and compete or cross-compete for binding to human PD-1 with, or bind to the same epitope on, human PD-1 as, nivolumab (see, e.g., U.S. Pat. Nos. 8,008,449 and 8,779,105; WO 2013/173223) or another PD-1 antagonist antibody. In some embodiments, PD-1/PD-L1 axis inhibitor antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the methods disclosed herein, a PD-1 antagonist antibody includes an antigen-binding portion or fragment that binds to the PD-1 receptor and exhibits the functional properties similar to those of whole antibodies in inhibiting ligand binding and upregulating the immune system. In certain embodiments, the PD-1 antagonist antibody or antigen-binding portion thereof cross-competes with nivolumab for binding to human PD-1. PD-1/PD-L1 inhibitors include, but are not limited to, nivolumab, pembrolizumab, atelozilumab, durvalumab, REGN2810, PDR001, AMP-514 (MEDI0608), AMP-224, BGB-A317 or a PD-1 or PD-L1 antagonist described in any one of the following publications: WO 2009/014708, WO 03/099196, WO 2009/114335 and WO 2011/161699.

In some embodiments, the immuno-oncology agent comprises a CTLA4 inhibitor. In certain embodiments, the CTLA4 inhibitor is an anti-CTLA4 antibody. In certain embodiments, the anti-CTLA4 antibody is ipilimumab. In certain embodiments, the immuno-oncology agent comprises an anti-CTLA4 inhibitor and a PD-1/PD-L1 axis inhibitor, wherein the PD-1/PD-L1 axis inhibitor comprises an anti-PD-1 or anti-PD-L1 antibody. In certain embodiments, the anti-CTLA4 antibody is ipilimumab and the anti-PD-1 antibody is nivolumab.

In some embodiments, the immuno-oncology agent comprises a TIGIT inhibitor (e.g., tiragolumab). In certain embodiments, the immuno-oncology agent comprises a TIGIT inhibitor and a PD-1/PD-L1 axis inhibitor, wherein the PD-1/PD-L1 axis inhibitor comprises an anti-PD-1 or anti-PD-L1 antibody.

In some embodiments, the immuno-oncology agent comprises a VISTA inhibitor (e.g., CI-8993). In certain embodiments, the immuno-oncology agent comprises a VISTA inhibitor and a PD-1/PD-L1 axis inhibitor, wherein the PD-1/PD-L1 axis inhibitor comprises an anti-PD-1 or anti-PD-L1 antibody.

In some embodiments, the immuno-oncology agent comprises a LAG-3 inhibitor (e.g., REGN3767). In certain embodiments, the immuno-oncology agent comprises a LAG-3 inhibitor and a PD-1/PD-L1 axis inhibitor, wherein the PD-1/PD-L1 axis inhibitor comprises an anti-PD-1 or anti-PD-L1 antibody. In some embodiments, the immuno-oncology agent comprises a CD73 inhibitor (e.g., SHR170008). In certain embodiments, the immuno-oncology agent comprises CD73 inhibitor and a PD-1/PD-L1 axis inhibitor, wherein the PD-1/PD-L1 axis inhibitor comprises an anti-PD-1 or anti-PD-L1 antibody.

In some embodiments, the immuno-oncology agent comprises a CD137 agonist (e.g., urelumab).

In some embodiments, the immuno-oncology agent comprises an OX40 agonist (e.g., MEDI-6383, MEDI-6469 or MOXR0916).

In some embodiments, the immuno-oncology agent comprises a CD40 agonist (e.g., lucatumumab).

In some embodiments, the immuno-oncology agent comprises a CD27 agonist (e.g., varlilumab).

In some embodiments, the immuno-oncology agent comprises a TLR7 agonist (e.g., resiquimod).

In some embodiments, the immuno-oncology agent comprises an interferon alpha polypeptide (e.g., INTRON A).

In some embodiments, the immuno-oncology agent comprises an IL-2 polypeptide (e.g., aldesleukin).

In some embodiments, the menin inhibitor reduces HOXA9 expression, MEIS1 expression, or a combination of both HOXA9 expression and MEIS1 expression.

In some embodiments, the cancer comprises a solid tumor. In some embodiments, the cancer comprises a liquid cancer, such as a hematological cancer, e.g., leukemia. In some embodiments, the tumor is or the cancer comprises breast cancer, bladder cancer, cervical cancer, colon cancer, head and neck cancer, liver cancer, lung cancer, kidney cancer, renal cell cancer, skin cancer, stomach cancer, prostate cancer, cholangiocarcinoma, brain cancer, Esophageal cancer, pancreatic cancer, or rectal cancer. In some embodiments, a therapeutically effective amount of the menin inhibitor is administered. In some embodiments, the menin inhibitor and the immuno-oncology agent are concurrently administered. In some embodiments, the menin inhibitor and the immuno-oncology agent are sequentially administered. In some embodiments, the menin inhibitor and the immuno-oncology agent are administered in separate dosage forms.

### Menin Inhibitors

In some embodiments, the menin inhibitor is a compound of Formula (I-A):

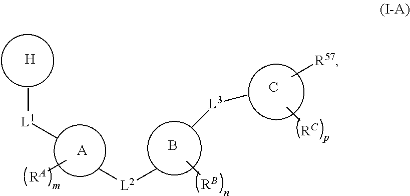

or a stereoisomer, tautomer, or isotopolog thereof, or a prodrug of any of the foregoing, or a pharmaceutically acceptable salt or any of the foregoing, or a solvate of any of the foregoing (collectively, pharmaceutically acceptable forms thereof), wherein:
H is selected from C₅₋₁₂ carbocycle and 5- to 12-membered heterocycle, each of which is optionally substituted with one or more R⁵⁰.
A is selected from bond, C₃₋₁₂ carbocycle and 3- to 12-membered heterocycle;
B is selected from C₃₋₁₂ carbocycle and 3- to 12-membered heterocycle;
C is 3- to 12-membered heterocycle;
L¹, L², and L³ are each independently selected from bond, —O—, —S—, —N(R⁵¹)—, —N(R⁵¹)CH₂—, —C(O)—, —C(O)O—, —OC(O)—, —OC(O)O—, —C(O)N(R⁵¹)—, —C(O)N(R⁵¹)C(O)—, —C(O)N(R⁵¹)C(O)N(R⁵¹)—, —N(R⁵¹)C(O)—, —N(R⁵¹)C(O)N(R⁵¹)—, —N(R⁵¹)C(O)O—, —OC(O)N(R⁵¹)—, —C(NR⁵¹)—, —N(R⁵¹)C(NR⁵¹)—, —C(NR⁵¹)N(R⁵¹)—, —N(R⁵¹)C(NR⁵¹)N(R⁵¹)—, —S(O)₂—, —OS(O)—, —S(O)O—, —S(O)—, —OS(O)₂—, —S(O)₂O—, —N(R⁵¹)S(O)₂—, —S(O)₂N(R⁵¹)—, —N(R⁵¹)S(O)—, —S(O)N(R⁵¹)—, —N(R⁵¹)S(O)₂N(R⁵¹)—, and —N(R⁵¹)S(O)N(R⁵¹)—; and alkylene, alkenylene, alkynylene, heteroalkylene, heteroalkenylene, and heteroalkynylene, each of which is optionally substituted with one or more R⁵⁰, wherein two R⁵⁰ groups attached to the same atom or different atoms of any one of L¹, L² or L can together optionally form a bridge or ring;
R^{A}, R^{B}, and R^{C} are each independently selected at each occurrence from R⁵⁰; or two R^{A} groups, two R^{B} groups or two R^{C} groups attached to the same atom or different atoms can together optionally form a bridge or ring;
m, n, and p are each independently an integer from 0 to 6;
R⁵⁰ is independently selected at each occurrence from:
   halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴—C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), and —P(O)(NR⁵²)₂; or two R⁵⁰ groups attached to the same atom taken together form ═O, ═S, or ═N(R⁵²);
   C₁₋₁₀ alkyl, C₂₋₁₀ alkenyl, and C₂₋₁₀ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₁₂ carbocycle and 3- to 12-membered heterocycle in R⁵⁰ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵¹ is independently selected at each occurrence from:
   hydrogen, —C(O)R⁵², —C(O)OR⁵², —C(O)N(R⁵²)₂, —C(O)NR⁵³R⁵⁴;
   C₁₋₆ alkyl, C₂₋₆ alkenyl, and C₂₋₆ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₁₂ carbocycle and 3- to 12-membered heterocycle in R⁵¹ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵² is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R⁵³ and R⁵⁴ are taken together with the nitrogen atom to which they are attached to form a heterocycle; and
R⁵⁷ is selected from:
   halogen, —NO₂, —CN, —SR⁵², —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)OR⁵², —OC(O)R⁵², —OC(O)OR⁵², —OC(O)N(R⁵²)₂, —OC(O)NR⁵³R⁵⁴, —NR⁵²C(O)OR⁵², —NR⁵²C(O)N(R⁵²)₂, —NR⁵²C(O)NR⁵³R⁵⁴, —C(O)NH(C₁₋₆ alkyl), —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═S, ═N(R⁵²); and
   C₁₋₁₀ alkyl, C₂₋₁₀ alkenyl, and C₂₋₁₀ alkynyl, each of which is substituted at each occurrence with one or more substituents independently selected from —NO₂, —CN, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —P(O)(OR⁵²)₂, —P(O)(R⁵²)₂, —P(O)(OR⁵²)(R⁵²)—P(O)(NR⁵²)(R⁵²), —NR⁵²P(O)(R⁵²), —P(O)(NR⁵²)(OR⁵²), —P(O)(NR⁵²)₂, ═S, and ═N(R⁵²);
wherein when C is azetidinylene, piperidinylene, or piperazinylene and R⁵⁷ is —S(═O)₂R⁵⁸, —
   S(═O)₂N(R⁵²)₂, or —NR⁵²S(═O)₂R⁵²;
   p is an integer from 1 to 6; and/or
   L³ is substituted with one or more R⁵⁰, wherein L³ is not —CH₂CH(OH)—.

In some embodiments, the menin inhibitor is a compound of Formula (I-B):

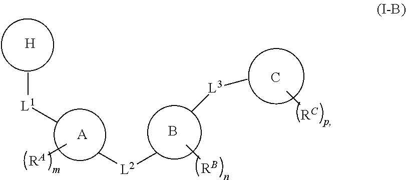

or a pharmaceutically acceptable form thereof, wherein:
H is selected from C₅₋₁₂ carbocycle and 5- to 12-membered heterocycle, each of which is optionally substituted with one or more R⁵⁰.
A, B, and C are each independently selected from C₃₋₁₂ carbocycle and 3- to 12-membered heterocycle;
L¹ and L² are each independently selected from bond, —O—, —S—, —N(R⁵¹)—, —N(R⁵¹)CH₂—, —C(O)—, —C(O)O—, —OC(O)—, —OC(O)O—, —C(O)N(R⁵¹)—, —C(O)N(R⁵¹)C(O)—, —C(O)N(R⁵¹)C(O)N(R⁵¹)—, —N(R⁵¹)C(O)—, —N(R⁵¹)C(O)N(R⁵¹)—, —N(R⁵¹)C(O)O—, —OC(O)N(R⁵¹)—, —C(NR⁵¹)—, —N(R⁵¹)C(NR⁵¹)—, —C(NR⁵¹)N(R⁵¹)—, —N(R⁵¹)C(NR⁵¹)N(R⁵¹)—, —S(O)₂—, —OS(O)—, —S(O)O—, —S(O)—, —OS(O)₂—, —S(O)₂O—, —N(R⁵¹)S(O)₂—, —S(O)₂N(R⁵¹)—, —N(R⁵¹)S(O)—, —S(O)N(R⁵¹)—, —N(R⁵¹)S(O)₂N(R⁵¹)—, and —N(R⁵¹)S(O)N(R⁵¹)—; and alkylene, alkenylene, alkynylene, heteroalkylene, heteroalkenylene, and heteroalkynylene, each of which is optionally substituted with one or more R⁵⁰.
L³ is selected from alkylene, alkenylene, and alkynylene, each of which is substituted with one or more R⁵⁶ and optionally further substituted with one or more R⁵⁰;
R^{A}, R^{B}, and R^{C} are each independently selected at each occurrence from R⁵⁰; or two R^{A} groups, two R^{B} groups or two R^{C} groups attached to the same atom or different atoms can together optionally form a bridge or ring;
m, n, and p are each independently an integer from 0 to 6;
R⁵⁰ is independently selected at each occurrence from:
   halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴—C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²), —P(O)(NR⁵²)(OR⁵²), and —P(O)(NR⁵²)₂, or two R⁵⁰ groups attached to the same atom taken together form ═O, ═S, ═N(R⁵²);
   C₁₋₁₀ alkyl, C₂₋₁₀ alkenyl, and C₂₋₁₀ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₁₂ carbocycle and 3- to 12-membered heterocycle in R⁵⁰ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵¹ is independently selected at each occurrence from:
   hydrogen, —C(O)R⁵², —C(O)OR⁵², —C(O)N(R⁵²)₂, —C(O)NR⁵³R⁵⁴;
   C₁₋₆ alkyl, C₂₋₆ alkenyl, and C₂₋₆ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²)—NR⁵²P(O)(R⁵²)—P(O)(NR⁵²)(OR⁵²), —P(O)(NR⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₁₂ carbocycle and 3- to 12-membered heterocycle in R⁵¹ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl;
R⁵² is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R⁵³ and R⁵⁴ are taken together with the nitrogen atom to which they are attached to form a heterocycle;
R⁵⁶ is independently selected at each occurrence from:
   —NO₂, —OR⁵⁹, —SR⁵², —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, C₁₋₁₀ alkyl, C₂₋₁₀ alkenyl, C₂₋₁₀ alkynyl, C₃₋₁₂ carbocycle, and 3- to 12-membered heterocycle, or two R⁵⁶ groups attached to the same atom taken together form ═O, ═S, or ═N(R⁵²);
   wherein each C₁₋₁₀ alkyl, C₂₋₁₀ alkenyl, and C₂₋₁₀ alkynyl in R⁵⁶ is optionally substituted at each occurrence with one or more substituents independently selected from halogen, —NO₂, —CN, —OR⁵⁹, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²)—NR⁵²P(O)(R⁵²)—P(O)(NR⁵²)(OR⁵²), —P(O)(NR⁵²)₂, ═O, ═S, ═N(R⁵²), C₃₋₁₂ carbocycle, and 3- to 12-membered heterocycle;
   wherein each C₃₋₁₂ carbocycle and 3- to 12-membered heterocycle in R⁵⁶ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, —P(O)(OR⁵²)(R⁵²), —P(O)(NR⁵²)(R⁵²), —NR⁵²P(O)(R⁵²)—P(O)(NR⁵²)(OR⁵²), —P(O)(NR⁵²)₂, ═O, ═S, ═N(R⁵²), C₁₋₆ alkyl, C₁₋₆ haloalkyl, C₂₋₆ alkenyl, and C₂₋₆ alkynyl; and
   further wherein R⁵⁶ optionally forms a bond to ring C; and
R⁵⁹ is independently selected at each occurrence from C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle,
wherein when R⁵⁶ is —CH₃, L³ is not further substituted with —OH, —NH₂, or —CN.

In some embodiments, for a compound of Formula (I-A) or (I-B), R^{C} is selected from —C(O)R⁵², —S(═O)R⁵², —S(═O)₂R⁵², —S(═O)₂N(R⁵²)₂, —S(═O)₂NR⁵³R⁵⁴, —NR⁵²S(═O)₂R⁵², =0, C₁₋₃ alkyl, and C₁₋₃ haloalkyl, or two R^{C} groups attached to different atoms can together form a C₁₋₃ bridge.

In some embodiments, the menin inhibitor is a compound of Formula (II-A):

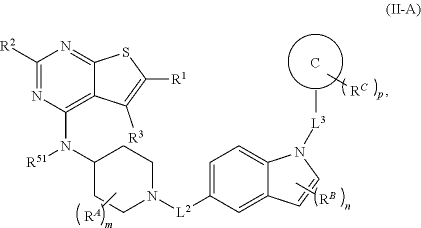

or a pharmaceutically acceptable form thereof, wherein:
C is selected from C₃₋₁₂ carbocycle and 3- to 12-membered heterocycle;
L² is selected from bond, —C(O)—, —C(O)O—, —C(O)N(R⁵¹)—, —C(O)N(R⁵¹)C(O)—, —C(O)N(R⁵¹)C(O)N(R⁵¹)—, —C(NR⁵¹)—, —S(O)₂—, —S(O)O—, —S(O)—, —S(O)₂O—, and —S(O)₂N(R⁵¹)—; and alkylene, alkenylene, alkynylene, heteroalkylene, heteroalkenylene, and heteroalkynylene, each of which is optionally substituted with one or more R⁵⁰;
L³ is selected from alkylene, alkenylene, and alkynylene, each of which is substituted with one or more R⁵⁶ and optionally further substituted with one or more R⁵⁰;
R¹ and R³ are each independently selected from hydrogen and R⁵⁰;
R² is R⁵⁰;
R^{A}, R^{B}, and R^{C} are each independently selected at each occurrence from R⁵⁰, or two R^{A} groups, two R^{B} groups, or two R^{C} groups attached to the same atom or different atoms can together optionally form a bridge or ring;
m, n, and p are each independently an integer from 0 to 6;
R⁵⁰ is independently selected at each occurrence from:
   halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴—C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, and —P(O)(R⁵²)₂, or two R⁵⁰ groups attached to the same atom taken together form ═O, ═S, or ═N(R⁵²)
   C₁₋₁₀ alkyl, C₂₋₁₀ alkenyl, and C₂₋₁₀ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₁₂ carbocycle and 3- to 12-membered heterocycle in R⁵⁰ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl;
R⁵¹ is independently selected at each occurrence from:
   hydrogen, —C(O)R⁵², —C(O)OR⁵², —C(O)N(R⁵²)₂, and —C(O)NR⁵³R⁵⁴; and
   C₁₋₆ alkyl, C₂₋₆ alkenyl, and C₂₋₆ alkynyl, each of which is optionally substituted at each occurrence with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle; and
   C₃₋₁₂ carbocycle and 3- to 12-membered heterocycle,
   wherein each C₃₋₁₂ carbocycle and 3- to 12-membered heterocycle in R⁵¹ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl;
R⁵² is independently selected at each occurrence from hydrogen; and C₁₋₂₀ alkyl, C₂₋₂₀ alkenyl, C₂₋₂₀ alkynyl, C₁₋₆ heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R⁵³ and R⁵⁴ are taken together with the nitrogen atom to which they are attached to form a heterocycle, optionally substituted with one or more R⁵⁰.
R⁵⁶ is independently selected at each occurrence from:
   —NO₂, —OR⁵⁹, —SR⁵², —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, C₁₋₁₀ alkyl, C₂₋₁₀ alkenyl, C₂₋₁₀ alkynyl, C₃₋₁₂ carbocycle, and 3- to 12-membered heterocycle, or two R⁵⁶ groups attached to the same atom taken together form ═O, ═S, or ═N(R⁵²);
   wherein each C₁₋₁₀ alkyl, C₂₋₁₀ alkenyl, and C₂₋₁₀ alkynyl in R⁵⁶ is optionally substituted at each occurrence with one or more substituents independently selected from halogen, —NO₂, —CN, —OR⁵⁹, —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₃₋₁₂ carbocycle, and 3- to 12-membered heterocycle;
   wherein each C₃₋₁₂ carbocycle and 3- to 12-membered heterocycle in R⁵⁶ is optionally substituted with one or more substituents independently selected from halogen, —NO₂, —CN, —OR⁵², —SR⁵², —N(R⁵²)₂, —NR⁵³R⁵⁴, —S(═O)R⁵², —S(═O)₂R⁵², —S(═O)₂N(R⁵²)₂, —S(═O)₂NR⁵³R⁵⁴, —NR⁵²S(═O)₂R⁵², —NR⁵²S(═O)₂N(R⁵²)₂, —NR⁵²S(═O)₂NR⁵³R⁵⁴, —C(O)R⁵², —C(O)OR⁵², —OC(O)R⁵², —OC(O)OR⁵², —OC(O)N(R⁵²)₂, —OC(O)NR⁵³R⁵⁴, —NR⁵²C(O)R⁵², —NR⁵²C(O)OR⁵², —NR⁵²C(O)N(R⁵²)₂, —NR⁵²C(O)NR⁵³R⁵⁴, —C(O)N(R⁵²)₂, —C(O)NR⁵³R⁵⁴, —P(O)(OR⁵²)₂, —P(O)(R⁵²)₂, ═O, ═S, ═N(R⁵²), C₁₋₆ alkyl, C₁₋₆ haloalkyl, C₂₋₆ alkenyl, and C₂₋₆ alkynyl; and
   further wherein R⁵⁶ optionally forms a bond to ring C; and
R⁵⁹ is independently selected at each occurrence from C₁₋₂₀ alkyl, C₂₋₂₀ alkenyl, C₂₋₂₀ alkynyl, C₁₋₆ heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle,
wherein when R⁵⁶ is —CH₃, L³ is not further substituted with —OH, —NH₂, or —CN.

In some embodiments, the menin inhibitor is a compound of Formula (III-A):

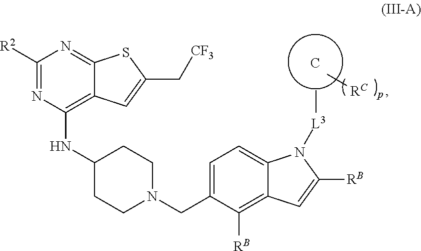

or a pharmaceutically acceptable form thereof, wherein R², R^{B}, R^{C}, L³, C, and p are each defined as described for Formula (II-A).

In some embodiments, the menin inhibitor is a compound of Formula (IV-A) or Formula (IV-B):

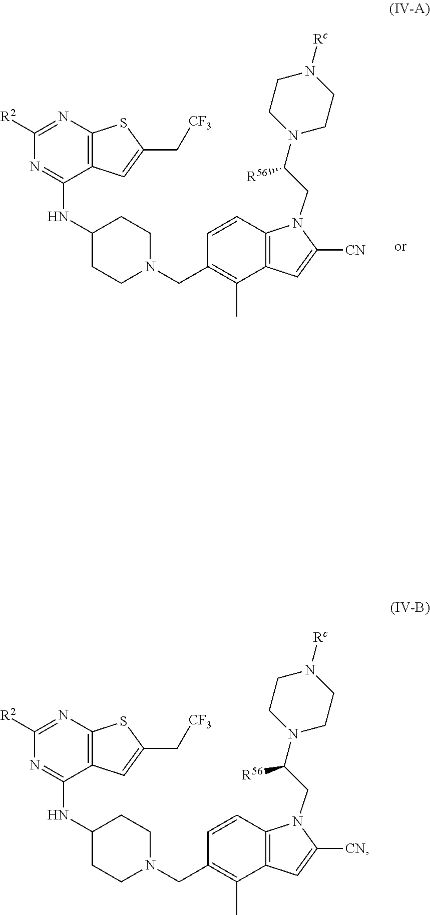

or a pharmaceutically acceptable form thereof, wherein R², R⁵⁶, and R^{C} are each defined as described for Formula (II-A).

In some embodiments, the menin inhibitor is Compound I:

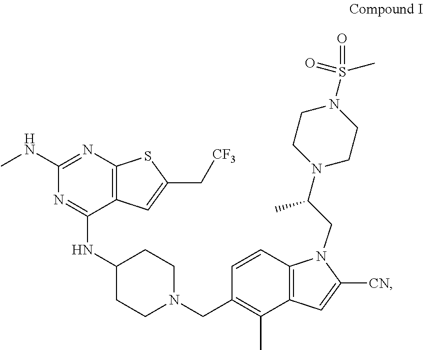

or a pharmaceutically acceptable form thereof, such as a pharmaceutically acceptable salt or solvate thereof.

In some embodiments, the menin inhibitor is a menin inhibitor described in U.S. Pat. No. 10,683,302, which disclosure is incorporated by reference herein. In some embodiments, the menin inhibitor is a compound of Formula (A-I):

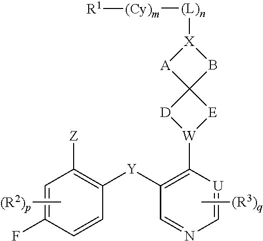

or a pharmaceutically acceptable form thereof, wherein:
A, B, D, and E are each independently selected from C(R^{A1})(R^{A2}), C(R^{A1})(R^{A2}) C(R^{A1})(R^{A2})—, —C(R^{A1})(R^{A2})—O—, —C(R^{A1})(R^{A2})—NR^{A3}—, C(═O)—, —C(R^{A1})(R^{A2})C(═O)—, and N═C(NH₂) wherein no more than one of A, B, D, and E is C(R^{A1})(R^{A2})—O—, C(R^{A1})(R^{A2})NR^{A3}—, —C(R^{A1})(R^{A2})C(═O)—, C(═O), or N═C(NH₂)—;
U is N or CR^{U}, wherein R^{U} is H, halo, CN, OH, C₁₋₄ alkyl, C₁₋₄ alkoxy, amino, C₁₋₄ alkyl amino, or C₂₋₈ dialkylamino;
W is N or CR^{W}, wherein R^{W} is H, halo, CN, OH, C₁₋₄ alkyl, C₁₋₄ alkoxy, amino, C₁₋₄ alkyl amino, or C₂₋₈ dialkylamino;
X is N or CR^{X}, wherein R^{X} is H, halo, CN, OH, C₁₋₄ alkyl, C₁₋₄ alkoxy, amino, C₁₋₄ alkyl amino, or C₂₋₈ dialkylamino, wherein when X is N, the atom of L that is directly bonded with X is other than N, O, or S;
L is selected from —C₁₋₆ alkylene- and —(C₁₋₄ alkylene)ₐ-Q-(C₁₋₄ alkylene)_{b}-, wherein the C₁₋₆ alkylene group and any C₁₋₄ alkylene group of the (C₁₋₄ alkylene)ₐ-Q-(C₁₋₄ alkylene)_{b}-group is optionally substituted with 1, 2, or 3 substituents independently selected from halo, CN, OH, C₁₋₃ alkyl, C₁₋₃ alkoxy, C₁₋₃ haloalkyl, C₁₋₃ haloalkoxy, amino, C₁₋₃ alkylamino, and di(C₁₋₃ alkyl)amino;
Q is —O—, —S—, —S(═O)—, S(═O)₂—, —C(═O)—, C(═O)NR^{q1}—, —C(═O)O—, —OC(═O)NR^{g1}—, —NR^{g1}—, —NR^{g1}C(═O)O—, —NR^{g1}C(═O)NR^{g1}—, S(═O)₂NR^{q1}—, —C(═NR^{q2})—, or —C(═NR^{q2})—NR^{g1}—, wherein each R^{q1} is independently selected from H or C₁₋₆ alkyl, and wherein each R^{q2} is independently selected from H, C₁₋₆ alkyl, and CN;
Cy is a linking C₆₋₁₄ aryl, C₃₋₁₈ cycloalkyl, 5-16 membered heteroaryl, or 4-18 membered heterocycle group, each of which is optionally substituted with 1, 2, 3, or 4 substituents independently selected from R^{Cy}, wherein the heteroaryl or heterocycle has 1-3 rings and 1-4 heteroatoms independently selected from nitrogen, sulfur, and oxygen;
each R^{Cy} is independently selected from halo, C₁₋₆ alkyl, C₁₋₄haloalkyl, C₁₋₄ cyanoalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycle, CN, NO₂, OR^{a1}, SR¹¹, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR¹S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycle are each optionally substituted by 1, 2, 3, or 4 substituents independently selected from CN, NO₂, OR^{a1}, SR¹¹, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1} NR^{c1}R^{d1}, NR^{c1}C(O)R^{b1} NR^{c1}C(O)OR^{a1}, NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}, wherein the heteroaryl or heterocycle has 1-3 rings and 1-4 heteroatoms independently selected from nitrogen, sulfur, and oxygen;
R¹ is H, Cy¹, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2} and S(O)₂NR^{c2}R^{d2}, wherein said C₁₋₆ alkyl, C₂₋₆ alkenyl, and C₂-6 alkynyl are each optionally substituted by 1, 2, 3, or 4 substituents independently selected from halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2} NR^{c2}C(O)OR^{a2} NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
Y is O, S, CR^{Y1}R^{Y2} or NR^{Y3}, wherein R^{Y1}, R^{Y2}, and R^{Y3} are each independently selected from H and C₁₋₄ alkyl;
Z is Cy², halo, C₁₋₆ alkyl, C₁₋₄haloalkyl, C₁₋₄ cyanoalkyl, C₂₋₆ alkenyl, C₂₋₆ alkynyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and P(O)R^{c3}R^{d3} wherein said C₁₋₆ alkyl, C₂₋₆ alkenyl, and C₂₋₆ alkynyl are each optionally substituted by 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3} NR^{c3}C(O)OR^{a3}, NR^{c3}C(O)NR^{c3}R^{d3} NR^{c3}S(O)R^{b3} NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R² and R³ is independently selected from H, halo, C₁₋₆ alkyl, C₁₋₄ haloalkyl, C₁₋₄ cyanoalkyl, C₂₋₆ alkenyl, C₂₋₆ alkynyl, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, and C₂₋₆ alkynyl are each optionally substituted by 1, 2, 3, or 4 substituents independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR⁴C(═NRe⁴)NR^{c4}R^{d4}, NR^{c4}R^{d4}, NR⁴C(O)R^{b4}, NR⁴C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4};
each R^{A1} is independently selected from H, halo, C₁₋₄ alkyl, C₁₋₄ alkoxy, C₁₋₄haloalkyl, C₁₋₄ haloalkoxy, amino, C₁₋₄ alkylamino, C₂₋₈ dialkylamino, CN, NO₂, and OH;
each R^{A2} is independently selected from H, halo, C₁₋₄ alkyl, C₁₋₄ alkoxy, C₁₋₄haloalkyl, C₁₋₄haloalkoxy, amino, C₁₋₄ alkylamino, C₂₋₈ dialkylamino, CN, NO₂, and OH;
each R^{A3} is independently selected from H, C₁₋₄ alkyl, C₁₋₄ alkoxy, C₁₋₄ haloalkyl, C(O)R^{z}, and C(O)OR^{z}, wherein said C₁₋₄ alkyl is optionally substituted by phenyl, C₁₋₄ alkoxy, C₁₋₄haloalkoxy, CN, NO₂, or OH;
R^{z} is H, C₁₋₄ alkyl, or phenyl;
each Cy¹ is independently selected from C₆₋₁₄ aryl, C₃₋₁₈ cycloalkyl, 5-16 membered heteroaryl, and 4-18 membered heterocycle, each of which is optionally substituted with 1, 2, 3, or 4 substituents independently selected from R^{Cy1}, wherein the heteroaryl or heterocycle has 1-3 rings and 1-4 heteroatoms independently selected from nitrogen, sulfur, and oxygen;
each Cy² is independently selected from C₆₋₁₄ aryl, C₃₋₁₈ cycloalkyl, 5-16 membered heteroaryl, and 4-18 membered heterocycle, each of which is optionally substituted with 1, 2, 3, or 4 substituents independently selected from R^{Cy2}, wherein the heteroaryl or has 1-3 rings and 1-4 heteroatoms independently selected from nitrogen, sulfur, and oxygen;
each R^{Cy1} and R^{Cy2} is independently selected from halo, C₁₋₆ alkyl, C₁₋₄haloalkyl, C₁₋₄ cyanoalkyl, C₂₋₆ alkenyl, C₂₋₆ alkynyl, phenyl, C₃₋₇ cycloalkyl, 5-6 membered heteroaryl, and 4-7 membered heterocycle, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5} NR^{c5}S(O)₂R^{b5} NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, phenyl, C₃₋₇ cycloalkyl, 5-6 membered heteroaryl, and 4-7 membered heterocycle are each optionally substituted by 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5} NR^{c5}C(O)OR^{a5}, NR^{c5}C(O)NR^{c5}R^{d5}NR^{c5}S(O)R^{b5} NR^{c5}S(O)₂R^{b5} NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein the heteroaryl or heterocycle has 1-3 rings and 1-4 heteroatoms independently selected from nitrogen, sulfur, and oxygen;
each R^{a1}, R^{b1}, R^{c1}, R^{d1}, R^{a2}, R^{b2}, R^{c2}, R^{d2}, R^{a3}, R^{b3}, R^{c3}, R^{d3}, R^{a4}, R^{b}, R^{c4}, R^{d4}, R^{a5}, R^{b5}, R^{c5}, and R^{d5} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycle, C₆₋₁₀ aryl-C₁₋₆ alkyl, C₃₋₁₀ cycloalkyl-C₁₋₆ alkyl, (5-10 membered heteroaryl)-C₁₋₆ alkyl, and (4-10 membered heterocycle)-C₁₋₆ alkyl, wherein said C₁₋₆ alkyl, C₂₋₆alkenyl, C₂₋₆ alkynyl, C₆₋₁₀ aryl, C₃₋₁₀ cycloalkyl, 5-10 membered heteroaryl, 4-10 membered heterocycle, C₆₋₁₀ aryl-C₁₋₆ alkyl, C₃₋₁₀ cycloalky-C₁₋₆ alkyl, (5-10 membered heteroaryl)-C₁₋₆ alkyl, and (4-10 membered heterocycle)-C₁₋₆ alkyl are each optionally substituted with 1, 2, 3, 4, or 5 substituents independently selected from R⁹, wherein the heteroaryl or heterocycle has 1-3 rings and 1-4 heteroatoms independently selected from nitrogen, sulfur, and oxygen;
each R^{e1}, R^{e2}, R^{e3}, R^{e4}, and R^{e5} is independently selected from H, C₁₋₄ alkyl, and CN;
each R^{g} is independently selected from the group consisting of OH, NO₂, CN, halo, C₁₋₆ alkyl, C₂₋₆ alkenyl, C₂₋₆ alkynyl, C₁₋₄haloalkyl, C₁₋₆ alkoxy, C₁₋₆haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁-6 alkylsulfinyl, C₁₋₆alkylsulfonyl, carboxy, aminocarbonyl, C₁₋₆alkylcarbonyl, and C₁-6 alkoxycarbonyl;
n is 0 or 1;
m is 0 or 1;
p is 0, 1, 2, or 3;
q is 0, 1, or 2;
a is 0 or 1; and
b is 0 or 1,
wherein any cycloalkyl or heterocycle group is optionally further substituted by 1 or 2 oxo groups.

In some embodiments, the menin inhibitor is SNDX-5613, which has the structure of:

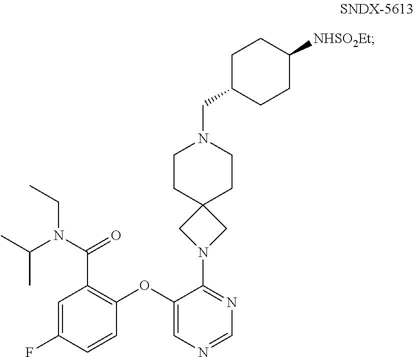

or a pharmaceutically acceptable form thereof.

In some embodiments, the menin inhibitor is VTP-50469, which has the structure of:

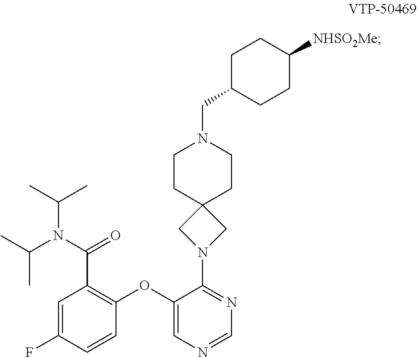

or a pharmaceutically acceptable form thereof.

In some embodiments, the menin inhibitor is a menin inhibitor described in U.S. Pat. Publ. No. 20210269454, which disclosure is incorporated by reference herein. In some embodiments, the menin inhibitor is a compound of Formula (A-II):

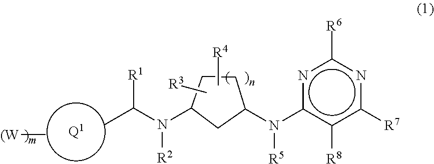

wherein
the dotted circle indicates that the ring is aromatic, R¹ and R² are each independently a hydrogen atom or a C₁₋₆ alkyl group, one of R³ and R⁴ is a hydrogen atom, a hydroxy group, a halogen atom, a C₁₋₆ alkoxy group, a di(C₁₋₆ alkyl)carbamoyl group, or an oxazolyl group, and the other of R³ and R⁴ is a hydrogen atom, a hydroxy group, a halogen atom, or a C₁₋₆ alkoxy group,
R⁵ is a hydrogen atom, a C₁₋₆ alkyl group, or a hydroxy C₁₋₆ alkyl group,
R⁶ is a hydrogen atom, a C₁₋₆ alkyl group, a halogen atom, a C₁₋₆ alkoxy group, an amino group, or a C₁₋₆ alkylamino group,
R⁷ and R⁸ are taken together with the carbon atom to which R⁷ is bonded and the carbon atom to which R⁸ is bonded to form any of the following formulas (2A) to (2C):

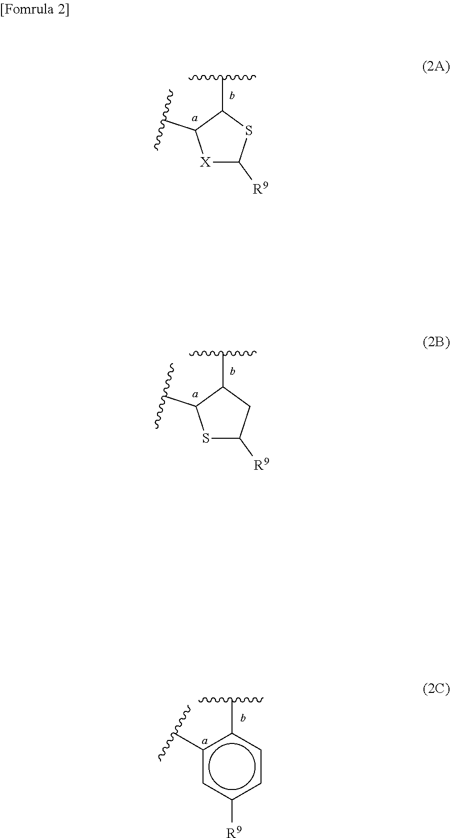

wherein
the dotted circle indicates that the ring is aromatic,
the carbon atom marked with a is the carbon atom to which R⁸ is bonded,
the carbon atom marked with b is the carbon atom to which R⁷ is bonded,
X is CH or a nitrogen atom, and
R⁹ is a halogen, C₁₋₆ alkyl group, a C₃₋₈ cycloalkyl group, a
C₃₋₈ cycloalkyl C₁₋₆ alkyl group, a C₁₋₆ alkoxy C₁₋₆ alkyl group, or an oxetanyl group, or
   R⁷ is a hydrogen atom, and R⁸ is the following formula (3):

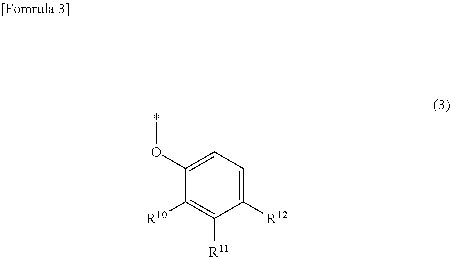

wherein
* indicates a bonding site,
R¹⁰ is a di(C₁₋₆ alkyl) carbamoyl group, a (C₁₋₆ alkyl)pyrimidinyl group, a (C₁₋₆ alkyl)phenyl group, or a (C₁₋₆ alkyl)pyrazolyl group,
R¹¹ is a hydrogen atom or a halogen atom, and
R¹² is a halogen atom,
m is 1 or 0,
n is 1 or 2,
Ring Q¹ is a 6-membered aromatic ring optionally containing one nitrogen atom in the ring (the aromatic ring optionally has one or two substituents independently selected from the following Group A), a 5-membered aromatic heterocycle containing, in the ring, one or two heteroatoms independently selected from the group consisting of a nitrogen atom and a sulfur atom (the aromatic heterocycle optionally has one substituent independently selected from the following Group A), a C₃₋₈ cycloalkane ring optionally having one substituent independently selected from the following Group A, a C₄₋₈ cycloalkene ring optionally having one substituent independently selected from the following Group A, a 4- to 8-membered saturated heterocycle containing one nitrogen atom in the ring (the saturated heterocycle optionally has one substituent independently selected from the following Group A), or a 9-membered bicyclic aromatic heterocycle containing one nitrogen atom in the ring (the bicyclic aromatic heterocycle optionally has one or two substituents independently selected from the following Group B), and W is the following formula (4A) or (4B):

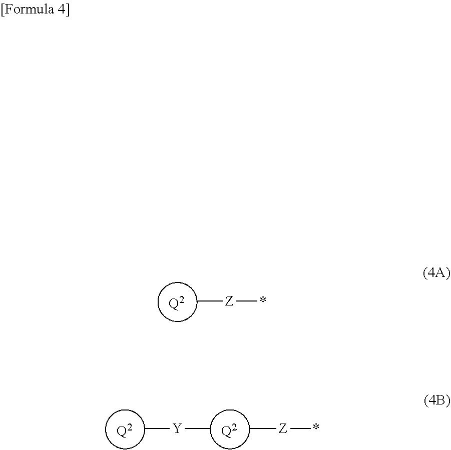

wherein
* indicates a bonding site,
Ring Q² is a 6-membered aromatic ring optionally containing one nitrogen atom in the ring (the aromatic ring optionally has one to three substituents independently selected from the following Group C), a 6-membered aromatic heterocycle containing two nitrogen atoms in the ring (the aromatic heterocycle optionally has one to three substituents independently selected from the following Group C), a 5-membered aromatic heterocycle containing, in the ring, one to three heteroatoms independently selected from the group consisting of a nitrogen atom, an oxygen atom and a sulfur atom (the aromatic heterocycle optionally has one substituent independently selected from the following Group C), a 9- or 10-membered bicyclic aromatic or partially unsaturated heterocycle containing, in the ring, one to three heteroatoms independently selected from the group consisting of a nitrogen atom and an oxygen atom (the bicyclic aromatic or partially unsaturated heterocycle optionally has one or two substituents independently selected from the following Group D), a 5- to 8-membered saturated heterocycle containing, in the ring, one or two heteroatoms independently selected from the group consisting of an oxygen atom and a nitrogen atom (the saturated heterocycle optionally has one substituent independently selected from the following Group E), or a C₃-8 cycloalkane ring optionally having one substituent independently selected from the following Group E, Ring Q³ is a 4- to 8-membered saturated heterocycle containing one nitrogen atom or one oxygen atom in the ring (the saturated heterocycle optionally has one C₁₋₆ alkylsulfonyl group), or a 6-membered aromatic ring optionally containing one nitrogen atom in the ring (the aromatic ring optionally has one substituent independently selected from the following Group F),
Y is a single bond or an oxygen atom, and
Z is a single bond, an oxygen atom, —NH—, —SO₂—, a C₁₋₆ alkylene group, *—R¹³—NHC(═O)—**, *—R⁴—O—**, or *—R⁵—NH—**,
wherein * is bonded to Ring Q², ** is bonded to Ring Q¹, and R¹³, R¹⁴ and R¹⁵ are each independently a C₁₋₆ alkylene group,
Group A: a halogen atom, a hydroxy group, a C₁₋₆ alkyl group, a C₁₋₆ alkoxy group, a hydroxy C₁₋₆ alkoxy group, a vinylsulfonylamino(C₁₋₆ alkyl)carbamoyl group, and a prop-2-enoylamino(C₁₋₆ alkyl)carbamoyl group,
Group B: a cyano group, a C₁₋₆ alkyl group, a halogen atom, and a C₁₋₆ alkoxy group,
Group C: a halogen atom, a C₁₋₆ alkyl group, a C₁₋₆ alkoxy group, a C₁₋₆ alkyl(C₁₋₆ alkylsulfonyl)amino group, a cyano group, a C₁₋₆ alkylsulfonyl group, a C₁₋₆ alkylamino group, a di(C₁₋₆ alkyl)amino group, a halogeno C₁₋₆ alkyl group, a C₁₋₆ alkoxy C₁₋₆ alkoxy group, a halogeno C₁₋₆ alkoxy group, a C₁₋₆ alkylsulfonyl C₁₋₆ alkyl group, a di(C₁-6 alkyl)sulfamoyl group, a C₁₋₆ alkylenedioxy group, a (C₁₋₆ alkyl)carbamoyl group, a hydroxy C₁₋₆ alkyl group, a 2-C₃₋₆ alkenoylamino group, a C₁₋₆ alkyl (2-C₃₋₆ alkenoyl)amino group, a hydroxy group, an oxo group, a —OC(²H)₃ group, and a —N[C(²H)₃]² group,
Group D: a halogen atom, a C₁₋₆ alkyl group, and a C₁₋₆ alkylsulfonyl group,
Group E: an oxo group, a hydroxy group, and a C₁₋₆ alkoxy group, and
Group F: a halogen atom, and a C₁₋₆ alkoxy group.

In some embodiments, the menin inhibitor is Compound A, which has the structure of:

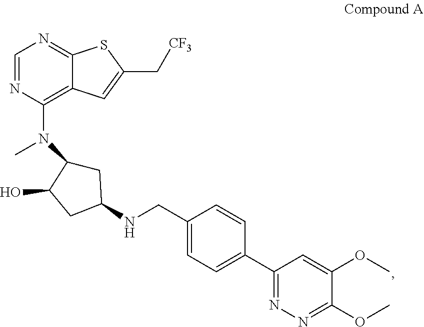 [SMILES: COc1cc(-c2ccc(CN[C@H]3C[C@@H](O)[C@@H](N(C)c4ncnc5sc(CC(F)(F)F)cc45)C3)cc2)nnc1OC]

or a pharmaceutically acceptable form thereof.

In some embodiments, the menin inhibitor is a menin inhibitor described in PCT Publ. No, WO2021/121327, which disclosure is incorporated by reference herein. In some embodiments, the menin inhibitor is a compound of Formula (A-III):

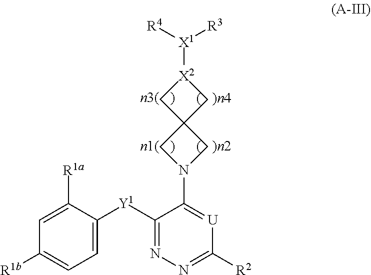

or a pharmaceutically acceptable form thereof, wherein
R^{1a} represents —C(═O)—NR^{xa}R^{xb}; Het or

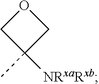

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}, —O— or —NR^{5c};
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N,
R⁴ represents isopropyl,
R^{5a}, R^{5b}, R^{5c}, R^{7a}, R^{7b} are each independently selected from the group consisting of hydrogen, C₁₋₄alkyl and C₃₋₆cycloalkyl,
R³ represents —C₁₋₆akyl-NR^{8a}R^{8b}, C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; C(═O)—C₁₋₄alkyl; —C(═O)—O—C₁₋₄alkyl; —C(═O)—NR^{12a}R^{12b}); and C₁₋₆alkyl substituted with one, two or three substituents each independently selected from the group consisting of —OH, cyano, halo, —S(═O)₂-C₁₋₄alkyl, —O—C₁₋₄alkyl, —C(═O)—NR^{10a}R^{10b}, and —NR^{10c}—C(═O)—C₁₋₄alkyl;
R^{9a}, R^{9b}, R^{10a}, R^{10b}, R^{10c}, R¹¹, R^{12a}, and R^{12b} are each independently selected from the group consisting of hydrogen and C₁₋₆alkyl.

In some embodiments, the menin inhibitor is Compound B, which has the structure of:

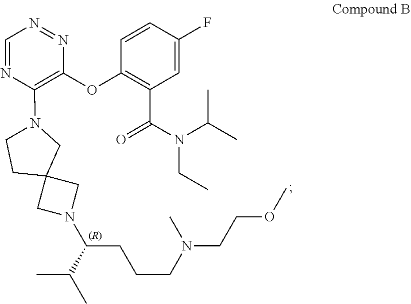

or a pharmaceutically acceptable form thereof.

In some embodiments, the menin inhibitor is a menin inhibitor described in U.S. Pat. No. 11,084,825, which disclosure is incorporated by reference herein. In some embodiments, the menin inhibitor is a compound of Formula (A-IV):

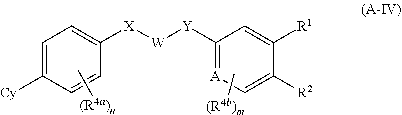

or a pharmaceutically acceptable form thereof,
wherein:
A is N;
Cy is:

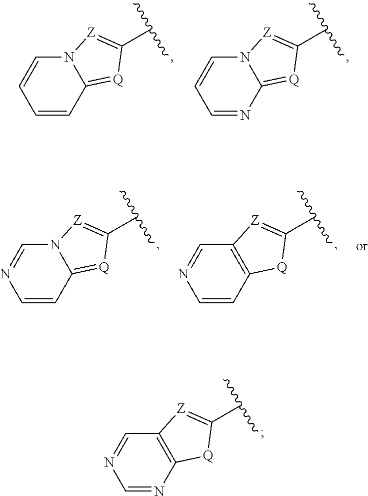

wherein:
Q is ═N—, —NH—, —O—, or —S—; and
Z is —CR^{5a}— or —N═;
wherein Cy is optionally substituted with one or more independently selected R⁷ substituents;
X is —C(R^{3b})₂—, —NR^{3a}—, or —O—;
W is —C(O)—, —S(O)—, or —S(O)₂—;
Y is a single bond, —C(R^{3b})₂—, —NR^{3a}—, or —O—;
(i) R¹ is H, halo, CN, C₁₋₆ alkyl, or C₁₋₆haloalkyl; and
R² is CH₂—Cy²-NHC(O)—C(R^{6a})═C(R^{6b})(R^{6c}) or Cy²-NHC(O)—C(R^{6a})═C(R^{6b})(R^{6c}); or
(ii) R¹ is CH₂—Cy²-NHC(O)—C(R^{6a})═C(R^{6b})(R^{6c}) or Cy²-NHC(O)—C(R^{6a})═C(R^{6b})(R^{6c}); and R² is H, halo, CN, C₁₋₆ alkyl, or C₁₋₆ haloalkyl;
each R^{3a} is independently H or C₁₋₆ alkyl;
each R^{3b} is independently H or C₁₋₆ alkyl;
each R^{4a} is independently H, halo, CN, C₁₋₆alkyl, C(O)R, C(O)N(R)₂, C(O)OR, N(R)₂, NRC(O)R, OR, S(O)₂R, C₃₋₇ cycloalkyl, a 4- to 7˜-membered heterocycloalkyl ring, phenyl, an 8- to 10-membered bicyclic aryl ring, or a 5- or 6-membered heteroaryl ring, wherein the 4- to 7-membered heterocycloalkyl ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur;
each R^{4b} is independently H, halo, CN, C₁₋₆alkyl, C(O)R, C(O)N(R)₂, C(O)OR, N(R)₂, NRC(O)R, OR, S(O)₂)R, C₃₋₇ cycloalkyl, a 4- to 7-membered heterocycloalkyl ring, phenyl, an 8a- to 10-membered bicyclic aryl ring, or a 5- or 6-membered heteroaryl ring, wherein the

In some embodiments, the menin inhibitor is a menin inhibitor described in PCT Publ. No. WO2021/121327, which disclosure is incorporated by reference herein. In some embodiments, the menin inhibitor is a compound of Formula (A-III):

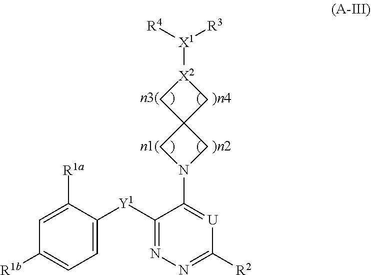

or a pharmaceutically acceptable form thereof, wherein
R^{1a} represents —C(═O)—NR^{xa}R^{xb}; Het; or

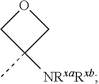

Het represents a 5- or 6-membered monocyclic aromatic ring containing one, two or three nitrogen atoms and optionally a carbonyl moiety;
wherein said 5- or 6-membered monocyclic aromatic ring is optionally substituted with one or two substituents selected from the group consisting of C₃₋₆cycloalkyl and C₁₋₄alkyl;
R^{xa} and R^{xb} are each independently selected from the group consisting of hydrogen, C₁₋₄alkyl and C₃₋₆cycloalkyl;
R^{1b} represents F or Cl;
Y¹ represents —CR^{5a}R^{5b}—, —O— or —NR^{5c};
R² is selected from the group consisting of hydrogen, halo, C₁₋₄alkyl, —O—C₁₋₄alkyl, and —NR^{7a}R^{7b};
U represents N or CH;
n1, n2, n3 and n4 are each independently selected from 1 and 2;
X¹ represents CH, and X² represents N;
R⁴ represents isopropyl;
R^{5a}, R^{5b}, R^{5c}, R^{7a}, and R^{7b}, are each independently selected from the group consisting of hydrogen, C₁₋₄alkyl and C₃₋₆cycloalkyl;
R³ represents —C₁₋₆alkyl-NR^{8a}R^{8b}, C₁₋₆alkyl-C(═O)—NR^{9a}R^{9b}, —C₁₋₆alkyl-OH, or —C₁₋₆alkyl-NR¹¹—C(═O)—O—C₁₋₄alkyl-O—C(═O)—C₁₋₄alkyl;
wherein each of the C₁₋₄alkyl or C₁₋₆alkyl moieties in the R³ definitions independently of each other may be substituted with one, two or three substituents each independently selected from the group consisting of cyano, halo, —OH, and —O—C₁₋₄alkyl;
R^{8a} and R^{8b} are each independently selected from the group consisting of hydrogen; C₁₋₆alkyl; —C(═O)—C₁₋₄alkyl; —C(═O)—O—C₁₋₄alkyl; —C(═O)—NR^{12a}R^{12b}); and C₁₋₆alkyl substituted with one, two or three substituents each independently selected from the group consisting of —OH, cyano, halo, —S(═O)₂-C₁₋₄alkyl, —O—C₁₋₄alkyl, —C(═O)—NR^{10a}R^{10b}, and —NR^{10c}—C(═O)—C₁₋₄alkyl;
R^{9a}, R^{9b}, R^{10a}, R^{10b}, R^{10c}, R¹¹, R^{12a}, and R^{12b} are each independently selected from the group consisting of hydrogen and C₁₋₆alkyl.

In some embodiments, the menin inhibitor is Compound B, which has the structure of:

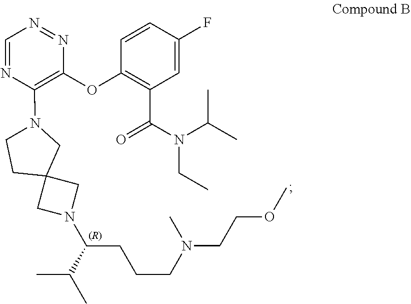

or a pharmaceutically acceptable form thereof.

In some embodiments, the menin inhibitor is a menin inhibitor described in U.S. Pat. No. 11,084,825, which disclosure is incorporated by reference herein. In some embodiments, the menin inhibitor is a compound of Formula (A-IV):

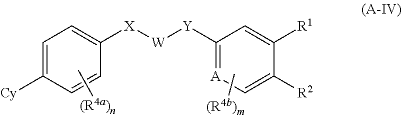

or a pharmaceutically acceptable form thereof,
wherein:
A is N;
Cy is:

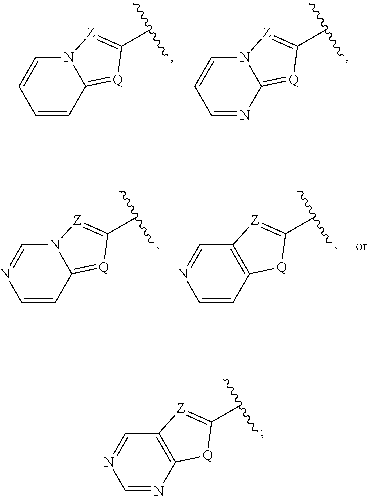

wherein:
Q is ═N—, —NH—, —O—, or —S—; and
Z is CR^{5a}═ or —N═;
wherein Cy is optionally substituted with one or more independently selected R⁷ substituents;
X is —C(R^{3b})₂—, —NR^{3a}—, or —O—;
W is —C(O)—, —S(O)—, or —S(O)₂—;
Y is a single bond, —C(R^{3b})₂—, —NR^{3a}—, or —O—;
(i) R¹ is H, halo, CN, C₁₋₆ alkyl, or C₁₋₆ haloalkyl; and
R² is CH₂—Cy²-NHC(O)—C(R^{6a})═C(R^{6b})═(R^{6c}) or Cy²-NHC(O)—C(R^{6a})═C(R^{6b})(R^{6c}); or
(ii) R¹ is CH₂-Cy²-NHC(O)—C(R^{6a})═C(R^{6b})(R^{6c}) or Cy²-NHC(O)—C(R^{6a})═C(R^{6b})(R^{6c}); and
R² is H, halo, CN, C₁₋₆ alkyl, or C₁₋₆ haloalkyl;
each R^{3a} is independently H or C₁₋₆ alkyl;
each R^{3b} is independently H or C₁₋₆ alkyl;
each R^{4a} is independently H, halo, CN, C₁₋₆ alkyl, C(O)R, C(O)N(R)₂, C(O)OR, N(R)₂, NRC(O)R, OR, S(O)₂R, C₃₋₇ cycloalkyl, a 4- to 7-membered heterocycloalkyl ring, phenyl, an 8- to 10-membered bicyclic aryl ring, or a 5- or 6-membered heteroaryl ring, wherein the 4- to 7-membered heterocycloalkyl ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur;
each R^{4b} is independently H, halo, CN, C₁₋₆ alkyl, C(O)R, C(O)N(R)₂, C(O)OR, N(R)₂, NRC(O)R, OR, S(O)₂R, C₃₋₇ cycloalkyl, a 4- to 7-membered heterocycloalkyl ring, phenyl, an 8- to 10-membered bicyclic aryl ring, or a 5- or 6-membered heteroaryl ring, wherein the 4- to 7-membered heterocycloalkyl ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur;
each R⁷ is independently a 4- to 7-membered heterocycloalkyl ring, phenyl, an 8- to 10-membered bicyclic aryl ring, or a 5- or 6-membered heteroaryl ring, wherein each 4- to 7-membered heterocycloalkyl ring independently has 1 or 2 heteroatoms independently selected from the group consisting of nitrogen, oxygen, and sulfur, and each 5- or 6-membered heteroaryl ring independently has 1, 2, 3, or 4 heteroatoms independently selected from the group consisting of nitrogen, oxygen, and sulfur, and further wherein each 4- to 7-membered heterocycloalkyl ring, phenyl, 8- to 10-membered bicyclic aryl ring, and 5- or 6-membered heteroaryl ring is optionally and independently substituted with one or more substituents independently selected from the group consisting of halo, CN, C₁₋₆ alkyl, C₁₋₆ haloalkyl, NH₂, NH(C₁₋₆ alkyl), N(C₁₋₆ alkyl)₂, OH, and O(C₁₋₆ alkyl);
each R is independently H, C₁₋₆ aliphatic, a saturated or partially unsaturated 4- to 7-membered heterocyclic ring, phenyl, an 8- to 10-membered bicyclic aryl ring, or a 5- or 6-membered heteroaryl ring, wherein the saturated or partially unsaturated 4- to 7-membered heterocyclic ring has 1 or 2 heteroatoms independently selected from the group consisting of nitrogen, oxygen, and sulfur, and the 5- or 6-membered heteroaryl ring has 1, 2, 3, or 4 heteroatoms independently selected from the group consisting of nitrogen, oxygen, and sulfur; or two geminal R groups, together with the nitrogen atom to which they are attached, form a saturated or partially unsaturated 4- to 7-membered heterocyclic ring or a 5- or 6-membered heteroaryl ring, wherein the 4- to 7-membered heterocyclic ring or the 5- or 6-membered heteroaryl ring has 0, 1, 2, or 3 additional heteroatoms independently selected from the group consisting of nitrogen, oxygen, and sulfur;
R^{5a} is H, halo, CN, C₁₋₆ alkyl, or C₁₋₆ haloalkyl;
R^{6a} is H or C₁₋₆ alkyl;
R^{6b} is H or C₁₋₆ alkyl; or
R^{6a} and R^{6b}, joined together, form a single bond;
R^{6c} is H or C₁₋₆ alkyl, wherein the C₁₋₆ alkyl is optionally substituted with N(CH₃)₂;
Cy² is a 4- to 7-membered heterocycloalkyl ring, phenyl, or pyridyl, wherein the 4- to 7-membered heterocycloalkyl ring has 1 or 2 heteroatoms independently selected from the group consisting of nitrogen, oxygen, and sulfur;
m is 1, 2, or 3; and
n is 1, 2, 3, or 4.

In some embodiments, the menin inhibitor is Compound C, which has the structure of:

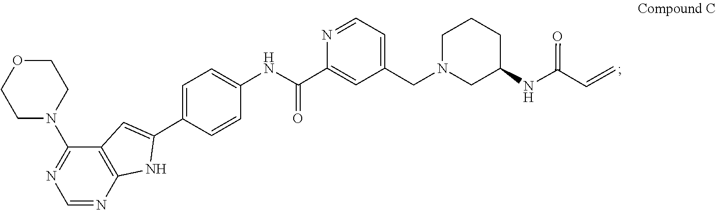 [SMILES: C=CC(=O)N[C@@H]1CCCN(Cc2ccnc(C(=O)Nc3ccc(-c4cc5c(N6CCOCC6)ncnc5[nH]4)cc3)c2)C1]

or a pharmaceutically acceptable form thereof.

In some embodiments, the menin inhibitor is Compound I, SNDX-5613, VTP-50469, Compound A, Compound B, or Compound C, or a pharmaceutically acceptable form thereof. In some embodiments, the menin inhibitor is Compound I, SNDX-5613, VTP-50469, Compound A, Compound B, or Compound C, or a pharmaceutically acceptable salt, stereoisomer, tautomer, prodrug, solvate, or isotopolog thereof.

In some embodiments, the menin inhibitor is Compound I, or a pharmaceutically acceptable salt, stereoisomer, tautomer, prodrug, solvate, or isotopolog thereof.

The compound of Formula (I-A), Formula (I-B), Formula (II-A), Formula (III-A), Formula (IV-A), or Formula (IV-B) (e.g., Compound I) may be synthesized by methods described in U.S. Pat. No. 10,781,218, which disclosure is incorporated by reference herein.

### Examples

### Example 1—MEN1 Function in A549 Lung Adenocarcinoma Cells

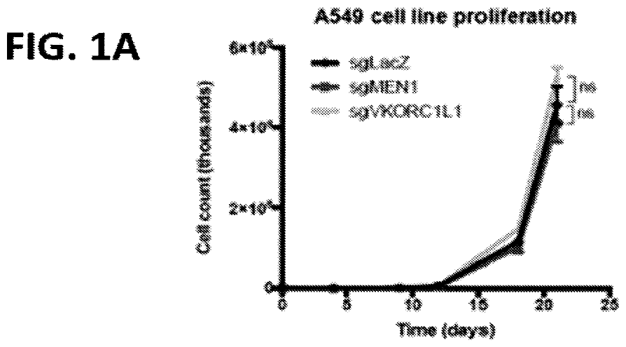

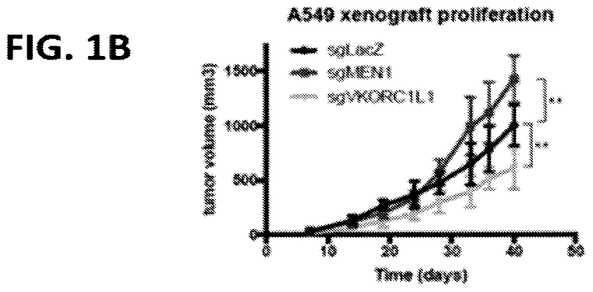

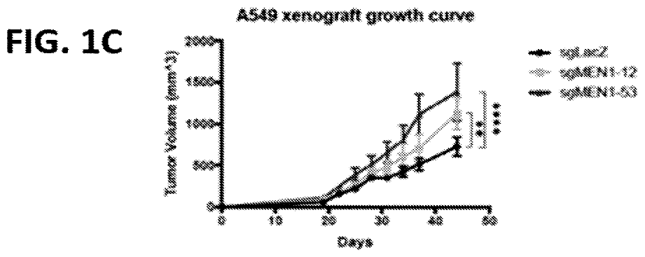

A549 cells with stable Cas9 expression were transduced with EpiDrug sgRNA library and selected with puromycin for 3 days. Cells were separated for an in vitro cell line screen and an in vivo xenograft screen, and studies were run in parallel for 3 weeks. Samples were collected for PCR amplification and Next Generation Sequencing. MEN1, the gene encoding the menin protein, was found to be enriched in the in vivo study but not in the in vitro study. CRISPR-mediated knockout of MEN1 was found to significantly increase growth of the human A549 cell line in vivo but not in vitro, showing that menin exerted its influence on tumor growth in this model via the tumor microenvironment (FIG. 1A-C).

### Example 2—MEN1 Regulation of Cytokine Gene Expression

RNA sequencing analysis of 2D cultured A549 cells with and without knockout of MEN1 identified the “cytokine-cytokine receptor interaction” pathway as the most significantly enriched pathway of upregulated genes in MEN1 knockout. The same pathway was identified as upregulated in MEN1 knockout A549 xenograft tumors. Examination of selected pathway genes, including CXCL1, IL33, CXCL8, and IL1B, also revealed upregulation in A549 MEN1 knockout cells. Examination of cytokine pathway genes in The Cancer Genome Atlas RNA-seq data set showed that genes in the “cytokine-cytokine receptor interaction” pathway were the top enriched group in patients with low MEN1 expression. These data suggest that MEN1 regulates expression of cytokine-related genes in cell line models and in patient tumors.

### Example 3—Mechanism of Cytokine Upregulation

MEN1 chromatin binding sites and MLL1 (with which MEN1 interacts) chromatin binding sites in A549 cells with and without knockout of MEN1 were identified using MACS and CREAM software. In MEN1 knockout cells, MLL1 occupancy was increased in certain broad peak regions common to both footprints. Analysis of the A549 RNA-seq data showed that most of the upregulated repeat regions in MEN1 knockout cells were located in the peak regions identified by CREAM analysis. Transcripts from repetitive genomic regions tend to form dsRNA; a significant induction of dsRNA staining was observed in MEN1 knockout cells compared to control A549 cells. These results suggest that MEN1 restricts MLL1 occupancy at repetitive genomic regions and loss of MEN1 activity leads to activation of dsRNA transcription.

MLL1 silencing experiments with siRNAs in A549 cells resulted in reduction of CXCL1, IL33, CXCL8, and IL1B in both control and MEN1 knockout cells, suggesting MEN1 regulation of cytokine-related genes is dependent on MLL1.

To determine whether MEN1 regulation of cytokine genes occurs through the viral mimicry mechanism, which depends on dsRNA sensing via the RIG-1/MDA5-MAVS pathway or reverse-transcribed DNA sensing via the cGAS-cGAMP STING pathway, MAVS or cGAS/STING were silenced in A549 cells with and without MEN1 knockout, leading to attenuation of cytokine induction in MEN1 knockout cells. These results suggest that MEN1 regulates cytokine-related genes through a MAVS and cGAS/STING dependent mechanism.

### Example 4—Correlation of MEN1 and MLL1 Expression with Immune Cell Infiltration

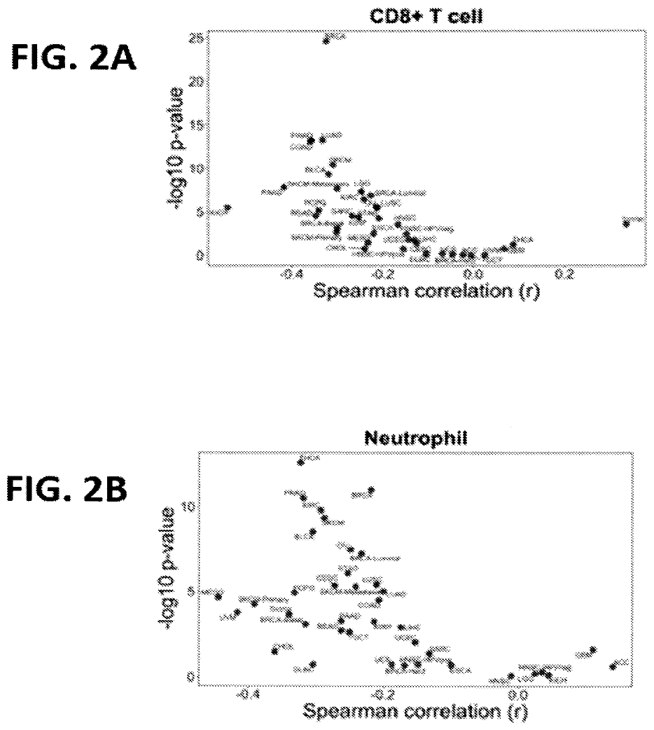

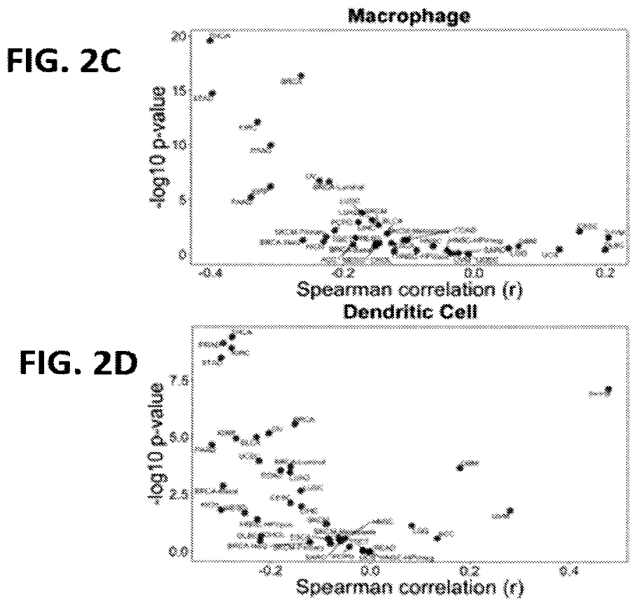

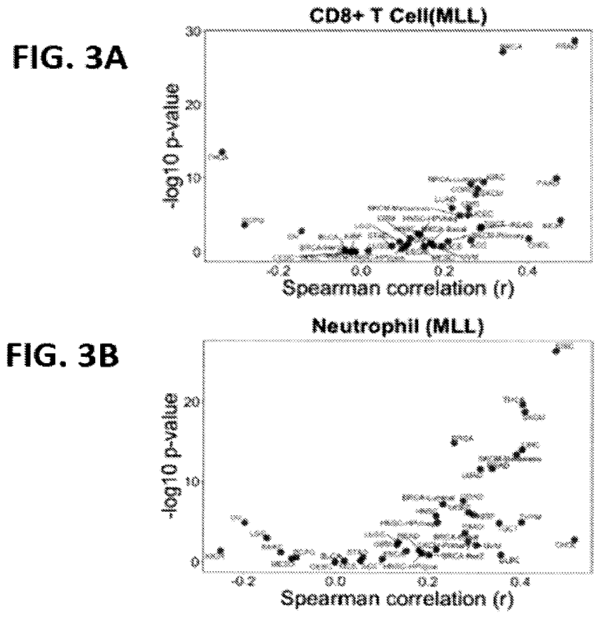

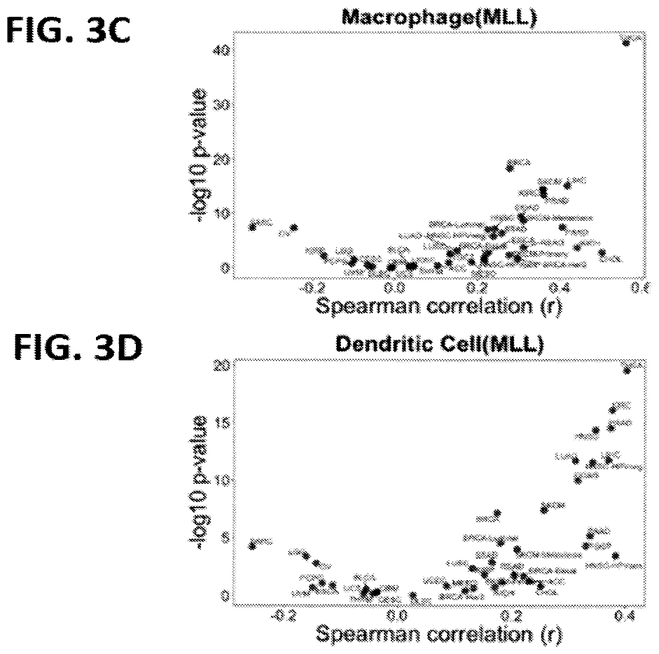

An immune cell infiltration analysis was performed on the TCGA data set using the TIMER software. An anti-correlation was observed between MEN1 abundance and neutrophil, CD8+, macrophage, and dendritic cell infiltration was observed in ˜70% of cancer types, including lung, colon, breast, prostate, kidney, and pancreatic cancers (FIGS. 2A-D). Strong positive correlation between MLL1 abundance and immune cell infiltration was observed in most cases (FIGS. 3A-D).

In A549 xenograft tumors, IHC staining with anti-myeloperoxidase (a marker from neutrophils), showed induction of neutrophil infiltration in MEN1 knockout tumors compared to control tumors. These data demonstrate that MEN1 depletion increases neutrophil infiltration within a tumor environment.

### Example 5—MEN1 Regulation of Tumor Growth in Cancer Models is Immune Status Dependent

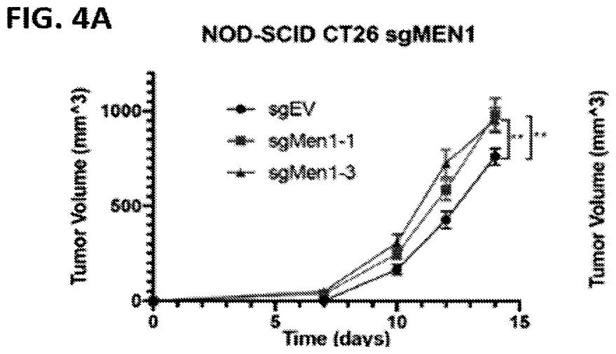

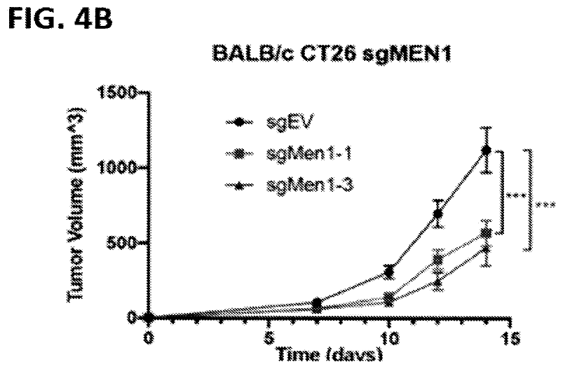

MEN1 deletion increased the growth rate of several murine solid tumors in immunodeficient animals, but paradoxically inhibited growth of the same tumors in immunocompetent animals. A murine-derived C26 colorectal carcinoma cell line was studied in immune-deficient NOD/SCID mice and immune-competent BALB/c mice. Genetic ablation of MEN1 did not alter CT26 cell proliferation in vitro but triggered faster tumor growth in immune-deficient mice (FIG. 4A). Loss of MEN1 resulted in significantly reduced tumor growth in immunocompetent mice (FIG. 4B).

Differential gene analysis of MEN1-proficient (control) and MEN1-deficient CT26 tumors in immunocompetent mice revealed activation of antiviral immune response pathways, upregulation of dsRNA species, and elevated expression and upregulation of cytokine-related genes such as CCL4, CXCL9, and CXCL10 in MEN1-deficient tumors, consistent with observations in A549 cells. These data demonstrate that MEN1 depletion in CT2 tumors activates dsRNA expression from repeat elements.

Transcriptomic analysis (scRNA-seq) of MEN1-proficient (control) and MEN1-deficient CT26 tumors grown in immunocompetent mice showed significantly increased levels (infiltration) of macrophages and T cells in MEN1 knockout tumors. Further profiling of immune cells by time-of-flight mass cytometry (CyTOF) showed an increase of percentage of immune cells (infiltration) in MEN1-deficient tumors, in particular an increase in CD8+ T cells, CD45+ cells, neutrophils, dendritic cells, and a subset of macrophages. These data demonstrate that MEN1 depletion in CT26 tumors promotes infiltration of immune cells such as CD8+ T cells and macrophages.

### Example 6—Compound I Induces Cytokine Gene Levels in Tumor Cells

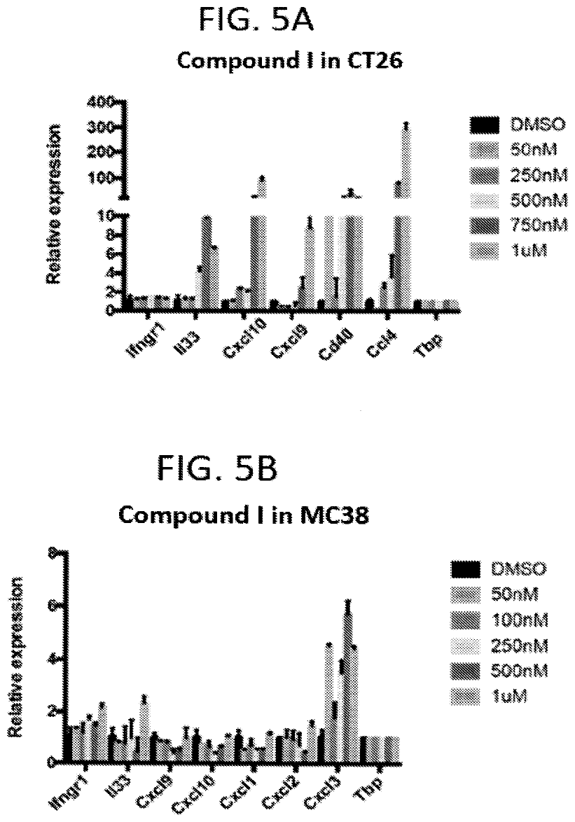

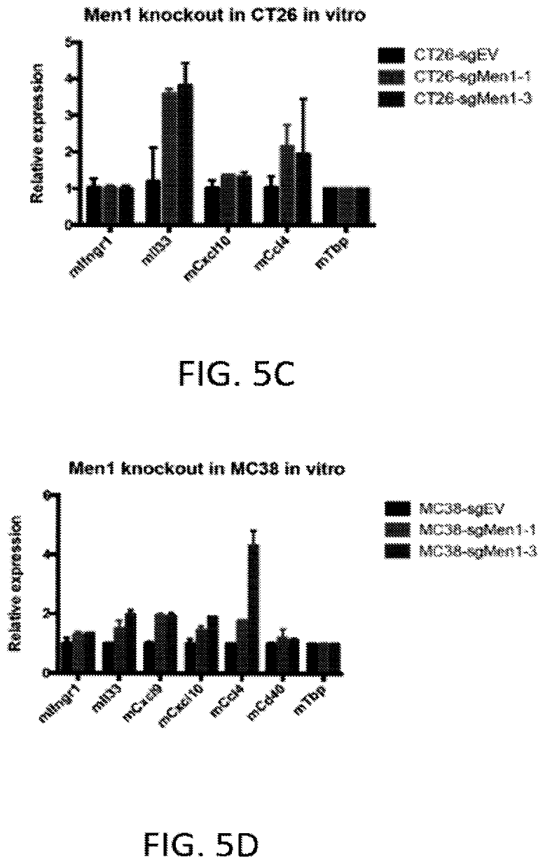

CT26 (immune-competent) and MC38 (immune-deficient) cancer cells were treated with Compound I. Dose-dependent induction of multiple cytokine-related genes, including IL33, CXCL10, CXCL9, CD40, and CCL4, was observed in CT26 cells (FIG. 5A-B). RT-qPCR analysis of 2D cultured CT26 and MC38 cells with and without knockout of MEN1 identified induction of cytokine-related genes, including IL33, CXCL10, CXCL9, CD40, and CCL4 (FIG. 5C-D).

### Example 7—Compound I Alone and in Combination with Immunotherapy Reduces Tumor Growth in Immunocompetent Mice

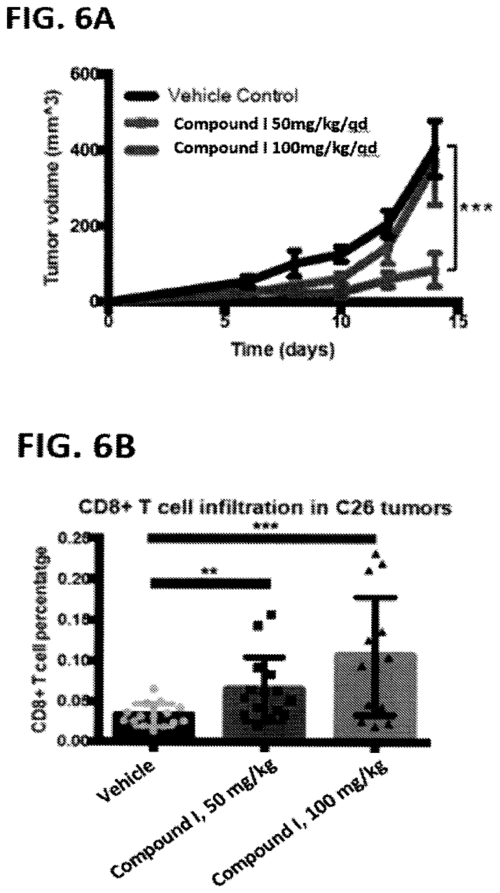

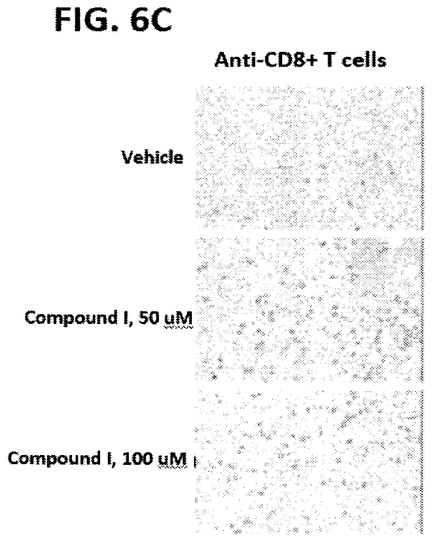

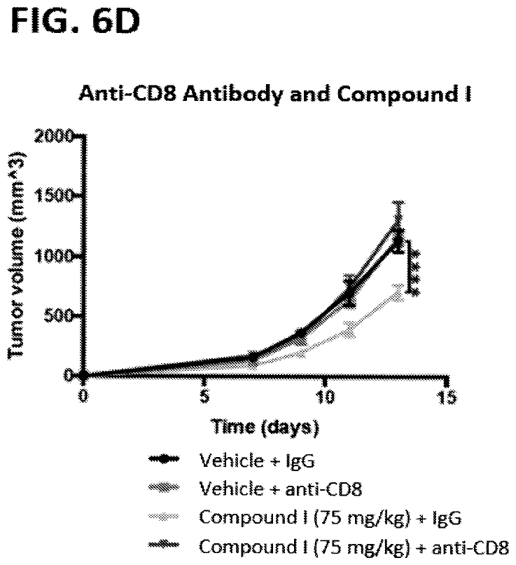

Treatment of immunocompetent (BALB/c) mice bearing CT26 colorectal carcinomas with Compound I at 100 mg/kg orally QD reduced tumor growth (FIG. 6A). Antitumor activity was associated with increased CD8+ T-cell infiltration (FIGS. 6B-C). The reduction in tumor growth could be blocked by concomitant administration of an anti-CD8 antibody (FIG. 6D).

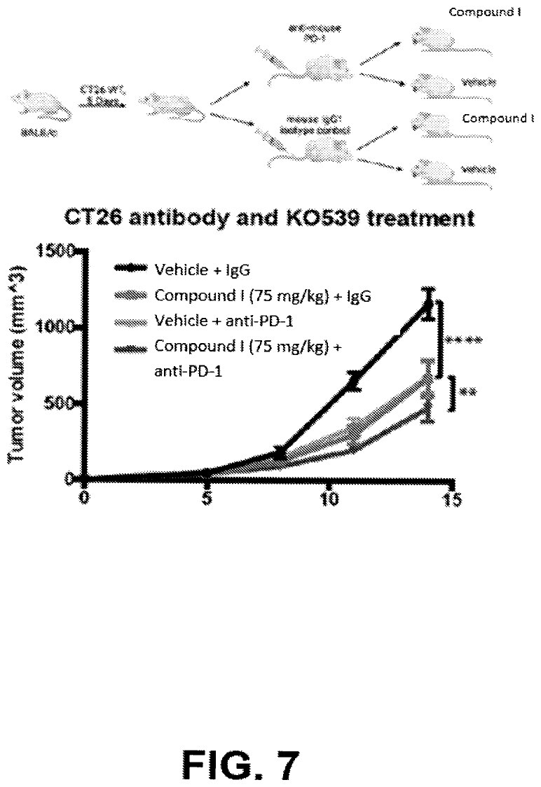

Effects of Compound I in combination with PD-L1/PD-1 immune checkpoint blockade on tumor growth were investigated in CT26 tumors. Compound I and an anti-PD-1 antibody each alone substantially reduced the CT26 tumor growth, and the combination enhanced the activity of anti-PD1 immunotherapy (FIG. 7).

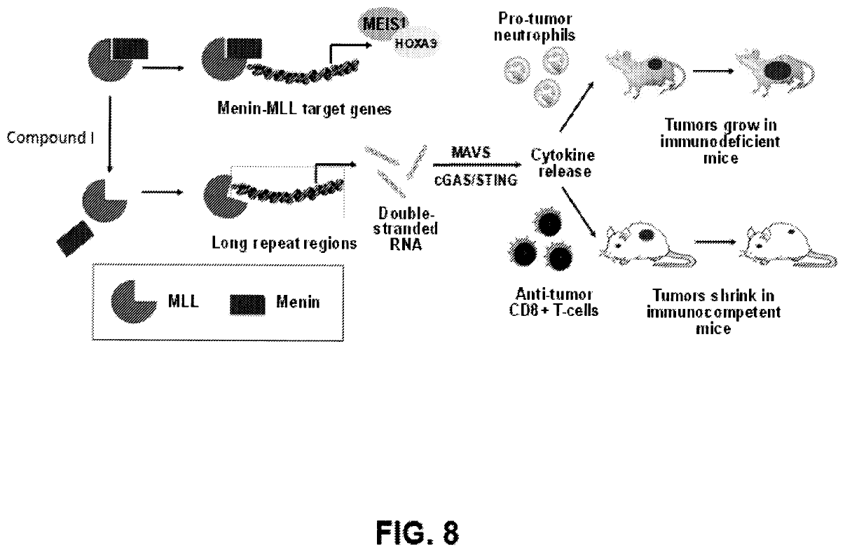

As shown in FIG. 8, disruption of the menin-MLL interaction by treatment with Compound I enhances T-cell-mediated antitumor immunity in solid tumor models. In certain instances, the mechanism underlying the advantageous effects include the release of the menin-MLL interaction (MLL (KMT2A) protein is generally bound to menin in COMPASS complexes) by a menin inhibitor such as Compound I, where MLL accumulates at different areas of chromatin from menin-HLL complexes, including long repeat regions harboring endogenous retroviral sequences. This leads to expression of double-stranded RNA (dsRNA) molecules that are recognized by the cGAS/STING sensor system, leading to activation of MAVS, which in turn increases production of a range of cytokines, including but not limited to CXCL1, 8, 9, 10 and 13, IL-33 and CCL4 (e.g., via increased expression of the corresponding genes and/or increased transcription). These cytokines can increase migration and activation of both tumor-promoting macrophages and neutrophils and tumor-suppressing CD8+ cytotoxic T cells. Hence, in immunodeficient mice lacking T cells, menin depletion stimulates tumor growth, whereas in immunocompetent animals the antitumor activity of T cells predominates, especially when combined with anti-PD1 immunotherapy.

FIGS. 1-8 and the discussion herein show and demonstrate through data: (i) the discovery that MEN1 functions in immune activation; (ii) that MEN1 depletion in immunocompetent models reduces tumor volume and increases immune activation (e.g., T cell and/or neutrophil cell infiltration/presence); (iii) the synergistic effect of combining a menin inhibitor (e.g., Compound I) and an immuno-oncology agent (e.g., a PD-1/PD-L1 axis inhibitor antibody) for methods of treating a tumor or cancer; and (iv) the means by which menin inhibitors can be evaluated for such immune-activating properties, alone or in combination with an immuno-oncology agent.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

1. A method of treating a tumor and/or cancer in an individual comprising administering to the individual a menin inhibitor and an immuno-oncology agent.

2. A method of enhancing an effect or a therapeutic effect (e.g., reduction in tumor size, efficacy, reduction in tumor cell growth or proliferation, etc.) of an immuno-oncology agent in an individual comprising administering to the individual the immuno-oncology agent and a menin inhibitor.

3. A method of activating or enhancing an immune response (e.g., increasing the presence, proliferation, or infiltration of immune cells, such as T cells, CD8+ T cells, CD4+ T cells, CD45+ cells, neutrophils, and/or macrophages, increasing expression and/or transcription of double-stranded RNA (dsRNA), increasing cytokine signaling (e.g., expression of cytokines such as CCL4, CXCL1, CXCL8, CXCL9, CXCL10, CD40, IL1B, or IL-33), etc.) in an individual, a cell, or a sample, comprising administering to the individual, or contacting the cell or sample, with a menin inhibitor, optionally in combination with an immuno-oncology agent, optionally wherein the individual has a tumor and/or a cancer.

4. The method of claim 1, claim 2, or claim 3, wherein the immuno-oncology agent comprises a PD-1/PD-L1 axis inhibitor, a CTLA4 inhibitor, a TIGIT inhibitor, a VISTA inhibitor, a LAG-3 (lymphocyte activation gene 3) inhibitor, a CD73 inhibitor, a CD137 (4-1BB) agonist, an OX40 agonist, a CD40 agonist, a CD27 agonist, a TLR7 agonist, an interferon alpha polypeptide, or an IL-2 polypeptide, or a combination thereof.

5. The method of claim 4, wherein the immuno-oncology agent comprises a PD-1/PD-L1 axis inhibitor, optionally wherein the PD-1/PD-L1 axis inhibitor comprises an anti-PD-1 or anti-PD-L1 antibody, optionally wherein the anti-PD-1 antibody or anti-PD-L1 antibody is nivolumab, pembrolizumab, cemiplimab, atezolizumab, dostarlimab, durvalumab, or avelumab.

6. The method of claim 4, wherein the immuno-oncology agent comprises a CTLA4 inhibitor, optionally wherein the CTLA4 inhibitor is an anti-CTLA4 antibody, optionally wherein the anti-CTLA4 antibody is ipilimumab.

7. The method of claim 6, wherein the immune-oncology agent comprises ipilimumab, and the immuno-oncology agent further comprises a PD-1/PD-L1 axis inhibitor, wherein the PD-1/PDL-1 axis inhibitor comprises an anti-PD-1 or anti-PD-L1 antibody, optionally wherein the anti-PD-1 antibody is nivolumab.

8. The method of claim 4, wherein the immuno-oncology agent comprises:
a TIGIT inhibitor (e.g., tiragolumab); or
a VISTA inhibitor (e.g., CI-8993); or
a LAG-3 inhibitor (e.g., REGN3767); or
a CD73 inhibitor (e.g., SHR170008);
optionally wherein the immuno-oncology agent further comprises a PD-1/PD-L1 axis inhibitor, wherein the PD-1/PD-L1 axis inhibitor comprises an anti-PD-1 or anti-PD-L1 antibody.

9. The method of claim 4, wherein the immuno-oncology agent comprises:
a CD137 agonist (e.g., urelumab); or
an OX40 agonist (e.g., MEDI-6383, MEDI-6469 or MOXR0916); or
a CD40 agonist (e.g., lucatumumab); or
a CD27 agonist (e.g., varlilumab); or
a TLR7 agonist (e.g., resiquimod); or
an interferon alpha polypeptide (e.g., INTRON A); or
an IL-2 polypeptide (e.g., aldesleukin).

10. The method of any one of claims 1 to 9, wherein the menin inhibitor, alone or in combination with the immuno-oncology agent:
increases a presence of (e.g., number of) immune cells in a tumor sample;
increases T-cell proliferation;
increases macrophage proliferation;
increases CD45+ cell proliferation;
increases T cell infiltration;
increases CD8+ T cell infiltration;
increases macrophage infiltration;
increases neutrophil infiltration;
increases expression and/or transcription of cytokine genes (e.g., CCL4, CXCL1, CXCL8, CXCL9, CXCL10, CD40, IL1B, or IL-33);
increases interferon-γ production;
increases cytokine (e.g,, IL-2) secretion;
increases an anti-tumor immune response within the tumor microenvironment (e.g., increased immune signaling);
increases expression and/or transcription of double stranded RNA recognized by cGAS/STING sensor system; and/or
inhibits tumor cell growth or reduces tumor volume in vivo.

11. The method of any one of claims 1 to 10, wherein the tumor is or the cancer comprises a solid tumor, optionally wherein the solid tumor is breast cancer, bladder cancer, cervical cancer, colon cancer, head and neck cancer, liver cancer, lung cancer, kidney cancer, renal cell cancer, skin cancer, stomach cancer, prostate cancer, pancreatic cancer, colon cancer, or rectal cancer.

12. The method of any one of claims 1 to 10, wherein the cancer comprises a liquid cancer, optionally wherein the liquid cancer is a hematological cancer, optionally wherein the hematological cancer is leukemia.

13. The method of any one of claims 1 to 12, wherein a therapeutically effective amount of the menin inhibitor is administered.

14. The method of any one of claims 1 to 13, wherein the menin inhibitor is Compound I: 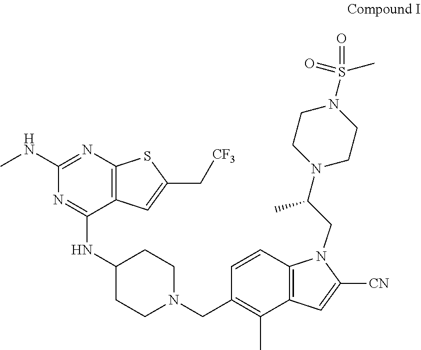
or a pharmaceutically acceptable salt or solvate thereof.
